SEQUENCE LISTING (2 sequences)

SEQ ID NO: 1 - DNA, 4760 bp, Homo sapiens
cttcctgaccggcgcgcgcagcctgctgccgcggtcagcgcctgctcctgctcctccgctcctcctgcgcggggtgctgaaacagcccggggaagtagagccgcctccggggagcccaaccagccgaacgccgccggcgtcagcagccttgcgcggccacagcatgaccgctcgcggcctggcccttggcctcctcctgctgctactgtgtccagcgcaggtgttttcacagtcctgtgtttggtatggagagtgtggaattgcatatggggacaagaggtacaattgcgaatattctggcccaccaaaaccattgccaaaggatggatatgacttagtgcaggaactctgtccaggattcttctttggcaatgtcagtctctgttgtgatgttcggcagcttcagacactaaaagacaacctgcagctgcctctacagtttctgtccagatgtccatcctgtttttataacctactgaacctgttttgtgagctgacatgtagccctcgacagagtcagtttttgaatgttacagctactgaagattatgttgatcctgttacaaaccagacgaaaacaaatgtgaaagagttacaatactacgtcggacagagttttgccaatgcaatgtacaatgcctgccgggatgtggaggccccctcaagtaatgacaaggccctgggactcctgtgtgggaaggacgctgacgcctgtaatgccaccaactggattgaatacatgttcaataaggacaatggacaggcaccttttaccatcactcctgtgttttcagattttccagtccatgggatggagcccatgaacaatgccaccaaaggctgtgacgagtctgtggatgaggtcacagcaccatgtagctgccaagactgctctattgtctgtggccccaagccccagcccccacctcctcctgctccctggacgatccttggcttggacgccatgtatgtcatcatgtggatcacctacatggcgtttttgcttgtgttttttggagcattttttgcagtgtggtgctacagaaaacggtattttgtctccgagtacactcccatcgatagcaatatagctttttctgttaatgcaagtgacaaaggagaggcgtcctgctgtgaccctgtcagcgcagcatttgagggctgcttgaggcggctgttcacacgctgggggtctttctgcgtccgaaaccctggctgtgtcattttcttctcgctggtcttcattactgcgtgttcgtcaggcctggtgtttgtccgggtcacaaccaatccagttgacctctggtcagcccccagcagccaggctcgcctggaaaaagagtactttgaccagcactttgggcctttcttccggacggagcagctcatcatccgggcccctctcactgacaaacacatttaccagccatacccttcgggagctgatgtaccctttggacctccgcttgacatacagatactgcaccaggttcttgacttacaaatagccatcgaaaacattactgcctcttatgacaatgagactgtgacacttcaagacatctgcttggcccctctttcaccgtataacacgaactgcaccattttgagtgtgttaaattacttccagaacagccattccgtgctggaccacaagaaaggggacgacttctttgtgtatgccgattaccacacgcactttctgtactgcgtacgggctcctgcctctctgaatgatacaagtttgctccatgacccttgtctgggtacgtttggtggaccagtgttcccgtggcttgtgttgggaggctatgatgatcaaaactacaataacgccactgcccttgtgattaccttccctgtcaataattactataatgatacagagaagctccagagggcccaggcctgggaaaaagagtttattaattttgtgaaaaactacaagaatcccaatctgaccatttccttcactgctgaacgaagtattgaagatgaactaaatcgtgaaagtgacagtgatgtcttcaccgttgtaattagctatgccatcatgtttctatatatttccctagccttggggcacatgaaaagctgtcgcaggcttctggtggattcgaaggtctcactaggcatcgcgggcatcttgatcgtgctgagctcggtggcttgctccttgggtgtcttcagctacattgggttgcccttgaccctcattgtgattgaagtcatcccgttcctggtgctggctgttggagtggacaacatcttcattctggtgcaggcctaccagagagatgaacgtcttcaaggggaaaccctggatcagcagctgggcagggtcctaggagaagtggctcccagtatgttcctgtcatccttttctgagactgtagcatttttcttaggagcattgtccgtgatgccagccgtgcacaccttctctctctttgcgggattggcagtcttcattgactttcttctgcagattacctgtttcgtgagtctcttggggttagacattaaacgtcaagagaaaaatcggctagacatcttttgctgtgtcagaggtgctgaagatggaacaagcgtccaggcctcagagagctgtttgtttcgcttcttcaaaaactcctattctccacttctgctaaaggactggatgagaccaattgtgatagcaatatttgtgggtgttctgtcattcagcatcgcagtcctgaacaaagtagatattggattggatcagtctctttcgatgccagatgactcctacatggtggattatttcaaatccatcagtcagtacctgcatgcgggtccgcctgtgtactttgtcctggaggaagggcacgactacacttcttccaaggggcagaacatggtgtgcggcggcatgggctgcaacaatgattccctggtgcagcagatatttaacgcggcgcagctggacaactatacccgaataggcttcgccccctcgtcctggatcgacgattatttcgactgggtgaagccacagtcgtcttgctgtcgagtggacaatatcactgaccagttctgcaatgcttcagtggttgaccctgcctgcgttcgctgcaggcctctgactccggaaggcaaacagaggcctcaggggggagacttcatgagattcctgcccatgttcctttcggataaccctaaccccaagtgtggcaaagggggacatgctgcctatagttctgcagttaacatcctccttggccatggcaccagggtcggagccacgtacttcatgacctaccacaccgtgctgcagacctctgctgactttattgacgctctgaagaaagcccgacttatagccagtaatgtcaccgaaaccatgggcattaacggcagtgcctaccgagtatttccttacagtgtgttttatgtcttctacgaacagtacctgaccatcattgacgacactatcttcaacctcggtgtgtccctgggcgcgatatttctggtgaccatggtcctcctgggctgtgagctctggtctgcagtcatcatgtgtgccaccatcgccatggtcttggtcaacatgtttggagttatgtggctctggggcatcagtctgaacgctgtatccttggtcaacctggtgatgagctgtggcatctccgtggagttctgcagccacataaccagagcgttcacggtgagcatgaaaggcagccgcgtggagcgcgcggaagaggcacttgcccacatgggcagctccgtgttcagtggaatcacacttacaaaatttggagggattgtggtgttggcttttgccaaatctcaaattttccagatattctacttcaggatgtatttggccatggtcttactgggagccactcacggattaatatttctccctgtcttactcagttacatagggccatcagtaaataaagccaaaagttgtgccactgaagagcgatacaaaggaacagagcgcgaacggcttctaaatttctagccctctcgcagggcatcctgactgaactgtgtctaagggtcggtcggtttaccactggacgggtgctgcatcggcaaggccaagttgaacaccggatggtgccaaccatcggttgtttggcagcagctttgaacgtagcgcctgtgaactcaggaatgcacagttgacttgggaagcagtattactagatctggaggcaaccacaggacactaaacttctcccagcctcttcaggaaagaaacctcattctttggcaagcaggaggtgacactagatggctgtgaatgtgatccgctcactgacactctgtaaaggccaatcaatgcactgtctgtctctccttttaggagtaagccatcccacaagttctataccatatttttagtgacagttgaggttgtagatacactttataacattttatagtttaaagagctttattaatgcaataaattaactttgtacacatttttatataaaaaaacagcaagtgatttcagaatgttgtaggcctcattagagcttggtctccaaaaatctgtttgaaaaaagcaacatgttcttcacagtgttcccctagaaaggaagagatttaattgccagttagatgtggcatgaaatgagggacaaagaaagcatctcgtaggtgtgtctactgggttttaacttatttttctttaataaaatacattgttttcctaagttttggggttaccctatctgctttgagagacaaatacaaaagctaaatggaagaga

SEQ ID NO: 2 - protein, 1278 aa, Homo sapiens
MTARGLALGLLLLLLCPAQVFSQSCVWYGECGIAYGDKRYNCEYSGPPKPLPKDGYDLVQELCPGFFFGNVSLCCDVRQLQTLKDNLQLPLQFLSRCPSCFYNLLNLFCELTCSPRQSQFLNVTATEDYVDPVTNQTKTNVKELQYYVGQSFANAMYNACRDVEAPSSNDKALGLLCGKDADACNATNWIEYMFNKDNGQAPFTITPVFSDFPVHGMEPMNNATKGCDESVDEVTAPCSCQDCSIVCGPKPQPPPPPAPWTILGLDAMYVIMWITYMAFLLVFFGAFFAVWCYRKRYFVSEYTPIDSNIAFSVNASDKGEASCCDPVSAAFEGCLRRLFTRWGSFCVRNPGCVIFFSLVFITACSSGLVFVRVTTNPVDLWSAPSSQARLEKEYFDQHFGPFFRTEQLIIRAPLTDKHIYQPYPSGADVPFGPPLDIQILHQVLDLQIAIENITASYDNETVTLQDICLAPLSPYNTNCTILSVLNYFQNSHSVLDHKKGDDFFVYADYHTHFLYCVRAPASLNDTSLLHDPCLGTFGGPVFPWLVLGGYDDQNYNNATALVITFPVNNYYNDTEKLQRAQAWEKEFINFVKNYKNPNLTISFTAERSIEDELNRESDSDVFTVVISYAIMFLYISLALGHMKSCRRLLVDSKVSLGIAGILIVLSSVACSLGVFSYIGLPLTLIVIEVIPFLVLAVGVDNIFILVQAYQRDERLQGETLDQQLGRVLGEVAPSMFLSSFSETVAFFLGALSVMPAVHTFSLFAGLAVFIDFLLQITCFVSLLGLDIKRQEKNRLDIFCCVRGAEDGTSVQASESCLFRFFKNSYSPLLLKDWMRPIVIAIFVGVLSFSIAVLNKVDIGLDQSLSMPDDSYMVDYFKSISQYLHAGPPVYFVLEEGHDYTSSKGQNMVCGGMGCNNDSLVQQIFNAAQLDNYTRIGFAPSSWIDDYFDWVKPQSSCCRVDNITDQFCNASVVDPACVRCRPLTPEGKQRPQGGDFMRFLPMFLSDNPNPKCGKGGHAAYSSAVNILLGHGTRVGATYFMTYHTVLQTSADFIDALKKARLIASNVTETMGINGSAYRVFPYSVFYVFYEQYLTIIDDTIFNLGVSLGAIFLVTMVLLGCELWSAVIMCATIAMVLVNMFGVMWLWGISLNAVSLVNLVMSCGISVEFCSHITRAFTVSMKGSRVERAEEALAHMGSSVFSGITLTKFGGIVVLAFAKSQIFQIFYFRMYLAMVLLGATHGLIFLPVLLSYIGPSVNKAKSCATEERYKGTERERLLNF

(19) United States
(12) Patent Application Publication
(10) Pub. No.: US 2022/0211687 A1
(43) Pub. Date: Jul. 7, 2022

(54) USE OF ARIMOCLOMOL IN THE TREATMENT OF NIEMANN PICK DISEASE

(71) Applicant: Orphazyme A/S, Copenhagen N (DK)

(72) Inventors: Thomas Kirkegaard JENSEN, Rødovre (DK); Christine í. DALI, Copenhagen K (DK); Nikolaj Havnsøe Torp PETERSEN, Copenhagen Ø (DK); Thomas BLAETTLER, Gelterkinden (CH); Lærke Clement FREIBERG, Kvistgård (DK)

(21) Appl. No.: 17/560,549

(22) Filed: Dec. 23, 2021

Related U.S. Application Data
(60) Provisional application No. 63/185,111, filed on May 6, 2021.
(60) Provisional application No. 63/130,438, filed on Dec. 24, 2020.

(51) Int. Cl. A61K 31/4545; A61P 43/00

(52) U.S. Cl. CPC A61K 31/4545; A61P 43/00

(57) ABSTRACT

This application is directed to assays, diagnostic methods and treatment methods using arimoclomol for the treatment of Niemann-Pick disease type C (NPC). More specifically, this application is directed to assays, diagnostics methods and treatment methods that target specific clinical subsets of patients with NPC who exhibit at least one missense mutation in at least one allele of an NPC gene.

### RELATED APPLICATIONS

This application claims priority to, and the benefit of, U.S. Provisional Application Nos. 63/130,438, filed on Dec. 24, 2020, and 63/185,111, filed on May 6, 2021, the contents of each of which are incorporated herein in their entirety.

### SEQUENCE LISTING

The instant application contains a Sequence Listing which has been submitted in ASCII format via EFS-Web and is hereby incorporated by reference in its entirety. Said ASCII copy, created on Dec. 23, 2021 is named “ORPH-009 001US SeqList.txt” and is about 18 KB in size.

### BACKGROUND

Niemann—Pick disease type C (NPC) is a rare, progressive, neurodegenerative disease in which lysosomal function is impaired and multiple lipid species accumulate in the lysosomal and endosomal compartments. This lipid accumulation leads to neurodegeneration and visceral organ dysfunction. The clinical presentation and progression of NPC is heterogeneous, depends on age at the time of neurological symptom onset, and includes loss of motor function, swallowing, and speech, as well as cognitive impairment. Individuals with infantile onset of neurological symptoms generally have a more aggressive disease course than patients with juvenile or late-onset disease.

The exact genetic and molecular mechanisms underlying NPC have been difficult to discern. Autosomal recessive mutations in either the NPC1 (-95% of cases) or NPC2 (-5% of cases) gene, which encode lysosomal proteins essential for the intracellular transport and metabolism of lipids, have been identified to be main genetic causes of NPC. However, there have been numerous different classes of mutations identified in NPC1 and NPC2 in patients with NPC, including missense mutations (70-80%) as well as splicing, frameshift, or premature stop mutations, which are collectively referred to herein as functional null mutations. Despite the identification of these mutations, consistent genotype/phenotype relationships in NPC have been difficult to establish. Moreover, different genotypes/phenotypes have differing responses to treatment, including treatment with miglustat, which has shown only a modest effect on slowing disease progression. Thus, there is a need in the art for genotype/patient specific treatments for NPC.

The present application addresses these needs by describing targeted treatments for subsets of NPC patients.

### SUMMARY

In some aspects, the present disclosure provides a method of treating or preventing Niemann Pick disease, type C (NPC) in a subject in need thereof, the method comprising administering a therapeutically effective amount of arimoclomol to the subject, wherein the subject has an NPC genotype of either Missense/Missense or Missense/Functional Null.

In some aspects, the present disclosure provides a method of treating or preventing Niemann Pick disease, type C (NPC) in a subject in need thereof, the method comprising administering a therapeutically effective amount of arimoclomol and a therapeutically effective amount of miglustat to the subject, wherein the subject has an NPC genotype of either Missense/Missense or Missense/Functional Null.

In some embodiments, the subject does not have an NPC genotype of Functional Null/Functional Null.

In some embodiments, the subject has a compound heterozygote NPC genotype.

In some aspects, the present disclosure provides a method of predicting the responsiveness of a subject with Niemann Pick disease, type C (NPC) to treatment with arimoclomol, the method comprising:

a) determining if the subject has an NPC genotype of:
i) Missense/Missense;
ii) Missense/Functional Null; or
iii) Functional Null/Functional Null; and

b) predicting that the subject will respond to treatment with arimoclomol when the subject is determined to have an NPC genotype of Missense/Missense or Missense/Functional Null or predicting that the subject will be less likely to respond to treatment with arimoclomol when the subject is determined to have an NPC genotype of Functional Null/Functional Null.

In some aspects, the present disclosure provides a method of predicting the responsiveness of a subject with Niemann Pick disease, type C (NPC) to treatment with a combination of arimoclomol and miglustat, the method comprising:

a) determining if the subject has an NPC genotype of:
i) Missense/Missense;
ii) Missense/Functional Null; or
iii) Functional Null/Functional Null; and

b) predicting that the subject will respond to treatment with a combination of arimoclomol and miglustat when the subject is determined to have an NPC genotype of Missense/Missense or Missense/Functional Null or predicting that the subject will be less likely to respond to treatment with a combination of arimoclomol and miglustat when the subject is determined to have an NPC genotype of Functional Null/Functional Null.

In some aspects, the present disclosure provides a method of identifying a subject with Niemann Pick disease, type C (NPC) who is likely to be responsive to treatment with arimoclomol, the method comprising:

a) determining if the subject has an NPC genotype of:
i) Missense/Missense;
ii) Missense/Functional Null; or
iii) Functional Null/Functional Null; and

b) identifying the subject as being likely to respond to treatment with arimoclomol when the subject is determined to have an NPC genotype of Missense/Missense or Missense/Functional Null or identifying the subject as less likely to respond to treatment with arimoclomol when the subject is determined to have an NPC genotype of Functional Null/Functional Null.

In some aspects, the present disclosure provides a method of identifying a subject with Niemann Pick disease, type C (NPC) who is likely to be responsive to treatment with a combination of arimoclomol and miglustat, the method comprising:

a) determining if the subject has an NPC genotype of:
i) Missense/Missense;
ii) Missense/Functional Null; or
iii) Functional Null/Functional Null; and

b) identifying the subject as being likely to respond to treatment with a combination of arimoclomol and miglustat when the subject is determined to have an NPC genotype of Missense/Missense or Missense/Functional Null or identifying the subject as less likely to respond to treatment with a combination arimoclomol and miglustat when the subject is determined to have an NPC genotype of Functional Null/Functional Null.

In some aspects, the present disclosure provides a method of treating or preventing Niemann Pick disease, type C (NPC) in a subject in need thereof, the method comprising:

a) determining if the subject has an NPC genotype of:
i) Missense/Missense;
ii) Missense/Functional Null; or
iii) Functional Null/Functional Null; and

b) administering a therapeutically effective amount of arimoclomol to the subject when the subject is determined to have an NPC genotype of Missense/Missense or Missense/Functional Null.

In some aspects, the present disclosure provides a method of treating or preventing Niemann Pick disease, type C (NPC) in a subject in need thereof, the method comprising:

a) determining if the subject has an NPC genotype of:
i) Missense/Missense;
ii) Missense/Functional Null; or
iii) Functional Null/Functional Null; and

b) administering a therapeutically effective amount of arimoclomol and a therapeutically effective amount of miglustat to the subject when the subject is determined to have an NPC genotype of Missense/Missense or Missense/Functional Null.

In some embodiments, the NPC genotype is an NPC1 genotype.

In some embodiments, the NPC genotype is an NPC2 genotype.

In some aspects, the present disclosure provides a method of treating or preventing Niemann Pick disease, type C (NPC) in a subject in need thereof, the method comprising administering a therapeutically effective amount of arimoclomol to the subject, wherein the subject is identified as having at least one missense mutation in at least one of the two alleles in an NPC gene.

In some aspects, the present disclosure provides a method of treating or preventing Niemann Pick disease, type C (NPC) in a subject in need thereof, the method comprising administering a therapeutically effective amount of arimoclomol and a therapeutically effective amount of miglustat to the subject, wherein the subject is identified as having at least one missense mutation in at least one of the two alleles in an NPC gene.

In some aspects, the present disclosure provides a method of predicting the responsiveness of a subject with Niemann Pick disease, type C (NPC) to treatment with arimoclomol, the method comprising:

a) determining the presence or absence of at least one missense mutation in at least one allele of an NPC gene; and

b) predicting that the subject will respond to treatment with arimoclomol when the presence of at least one missense mutation in at least one allele of an NPC gene is determined.

In some aspects, the present disclosure provides a method of predicting the responsiveness of a subject with Niemann Pick disease, type C (NPC) to treatment with a combination of arimoclomol and miglustat, the method comprising:

a) determining the presence or absence of at least one missense mutation in at least one allele of an NPC gene; and;

b) predicting that the subject will respond to treatment with a combination of arimoclomol and miglustat when the presence of at least one missense mutation in at least one allele of an NPC gene is determined.

In some aspects, the present disclosure provides a method of identifying a subject with Niemann Pick disease, type C (NPC) who will be responsive to treatment with arimoclomol, the method comprising:

a) determining the presence or absence of at least one missense mutation in at least one allele of an NPC gene; and;

b) identifying that the subject will be responsive to treatment with arimoclomol when the presence of at least one missense mutation in at least one allele of an NPC gene is determined.

In some aspects, the present disclosure provides a method of identifying a subject with Niemann Pick disease, type C (NPC) who will be responsive to treatment with a combination of arimoclomol and miglustat, the method comprising:

a) determining the presence or absence of at least one missense mutation in at least one allele of an NPC gene; and;

b) identifying that the subject will be responsive to treatment with a combination of arimoclomol and miglustat when the presence of at least one missense mutation in at least one allele of an NPC gene is determined.

In some aspects, the present disclosure provides a method of treating or preventing Niemann Pick disease, type C (NPC) in a subject in need thereof, the method comprising:

a) determining the presence or absence of at least one missense mutation in at least one allele of an NPC gene; and

b) administering a therapeutically effective amount of arimoclomol to the subject when the presence of at least one missense mutation in at least one allele of an NPC gene is determined.

In some aspects, the present disclosure provides a method of treating or preventing Niemann Pick disease, type C (NPC) in a subject in need thereof, the method comprising:

a) determining the presence or absence of at least one missense mutation in at least one allele of an NPC gene; and

b) administering a therapeutically effective amount of arimoclomol and a therapeutically effective amount of miglustat to the subject when the presence of at least one missense mutation in at least one allele of an NPC gene is determined.

In some embodiments, the subject is identified as having at least one missense mutation in each of the two alleles of the NPC gene.

In some embodiments, the subject is identified as having at least one missense mutation in one allele of the NPC gene and a functional null mutation in the other allele of the NPC gene.

In some embodiments, the subject is identified as having a functional null mutation in at most one allele of the two alleles of the NPC gene.

In some embodiments, the subject is identified as being a compound heterozygote for the NPC gene.

In some embodiments, the NPC gene is NPC1.

In some embodiments, the NPC gene is NPC2.

In some embodiments, determining the presence or absence of at least one missense mutation in at least one allele of the NPC gene comprises sequencing nucleic acid isolated from a biological sample from the subject.

In some embodiments, a functional null mutation is a mutation that results in a truncated and defective NPC protein being produced from the allele that has the functional null mutation.

In some embodiments, a functional null mutation is a frameshift mutation, an aberrant splicing mutation, or a premature stop codon mutation.

In some embodiments, the missense mutation results in a substitution, a deletion, an insertion, a duplication, or any combination thereof.

The details of the application are set forth in the accompanying description below. Although methods and materials similar or equivalent to those described herein can be used in the practice or testing of the present application, illustrative methods and materials are now described. Other features, objects, and advantages of the application will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this application belongs. All patents and publications cited in this specification are incorporated herein by reference in their entireties.

### BRIEF DESCRIPTION OF THE FIGURES

FIG. 1A, 1B, 1C, 1D and 1E show the observed changes of 5-domain NPCCSS scores as compared to baseline at month 12 following treatment with arimoclomol or placebo. FIG. 1A shows observed changes in 5-domain NPCCSS scores as compared to baseline in the entire population of subjects in the study. FIG. 1B shows observed changes in 5-domain NPCCSS scores as compared to baseline in subjects who were aged ≥4 years. FIG. 1C shows observed changes in 5-domain NPCCSS scores as compared to baseline in subjects who were also receiving miglustat. FIG. 1D shows observed changes in 5-domain NPCCSS scores as compared to baseline in subjects with either a missense/missense or missense/functional null NPC genotype (excluding patients double functional null mutations). In FIG. 1A, 1B, 1C and 1D, the solid line represents least-squares mean estimates ±standard error based on data obtained while subjects were exposed to study treatment. The mixed model for repeated measures included the main effect of baseline and stratum, respectively, and interaction between treatment and visit. Change from baseline and absolute estimates correspond to the at-baseline overall average subject. Numbers of subject are presented for each time point. FIG. 1E shows the patient-level change in 5-domain NPCCSS scores from baseline to last available data in all subjects in the study.

FIG. 2A, 2B and 2C are graphs of biomarker analyses in subjects administered arimoclomol or a placebo. FIG. 2A shows the change in HSP70 in PBMCs from months 0 to 12 in arimoclomol-treated patients. FIG. 2B shows the change in unesterified cholesterol level at month 12. FIG. 2C shows the change in serum cholestane-triol level at month 12 (between-group difference: p=0.225). Error bars show standard error.

### DETAILED DESCRIPTION

Arimoclomol is an orally available small molecule that crosses the blood—brain barrier, as evidenced by its presence in cerebrospinal fluid of treated patients with amyotrophic lateral sclerosis. In some embodiment, the present disclosure pertains, at least in part, to methods for treating Niemann Pick Type C disease (NPC) in specific clinical subsets of subjects using arimoclomol alone or a combination of arimoclomol and miglustat. The methods described herein are based, at least in part, on the surprising and unexpected finding, (described in the Exemplification section herein), that NPC subjects with at least one missense mutation in at least one of the two alleles in an NPC gene respond robustly to treatment with arimoclomol, in contrast with NPC subjects with a functional null mutation in both of the alleles of an NPC gene.

The present disclosure provides a method of treating or preventing Niemann Pick disease, type C (NPC) in a subject in need thereof, the method comprising administering a therapeutically effective amount of arimoclomol to the subject, wherein the subject has an NPC genotype of either Missense/Missense or Mi ssense/Functional Null.

The present disclosure also provides a method of treating or preventing Niemann Pick disease, type C (NPC) in a subject in need thereof, the method comprising administering a therapeutically effective amount of arimoclomol and a therapeutically effective amount of miglustat to the subject, wherein the subject has an NPC genotype of either Missense/Missense or Missense/Functional Null.

In some embodiments of the preceding methods, the subject does not have an NPC genotype of Functional Null/Functional Null.

In some embodiments of the preceding methods, the subject has a compound heterozygote NPC genotype.

The present disclosure provides a method of predicting the responsiveness of a subject with Niemann Pick disease, type C (NPC) to treatment with arimoclomol, the method comprising: a) determining if the subject has an NPC genotype of: i) Missense/Missense; ii) Missense/Functional Null; or iii) Functional Null/Functional Null; and b) predicting that the subject will respond to treatment with arimoclomol when the subject is determined to have an NPC genotype of Missense/Missense or Missense/Functional Null or predicting that the subject will be less likely to respond to treatment with arimoclomol when the subject is determined to have an NPC genotype of Functional Null/Functional Null.

The present disclosure also provides a method of predicting the responsiveness of a subject with Niemann Pick disease, type C (NPC) to treatment with a combination of arimoclomol and miglustat, the method comprising: a) determining if the subject has an NPC genotype of: i) Missense/Missense; ii) Missense/Functional Null; or iii) Functional Null/Functional Null; and b) predicting that the subject will respond to treatment with a combination of arimoclomol and miglustat when the subject is determined to have an NPC genotype of Missense/Missense or Missense/Functional Null or predicting that the subject will be less likely to respond to treatment with a combination of arimoclomol and miglustat when the subject is determined to have an NPC genotype of Functional Null/Functional Null.

The present disclosure provides a method of identifying a subject with Niemann Pick disease, type C (NPC) who is likely to be responsive to treatment with arimoclomol, the method comprising: a) determining if the subject has an NPC genotype of: i) Missense/Missense; ii) Missense/Functional Null; or iii) Functional Null/Functional Null; and b) identifying the subject as being likely to respond to treatment with arimoclomol when the subject is determined to have an NPC genotype of Missense/Missense or Missense/Functional Null or identifying the subject as less likely to respond to treatment with arimoclomol when the subject is determined to have an NPC genotype of Functional Null/Functional Null.

The present disclosure also provides a method of identifying a subject with Niemann Pick disease, type C (NPC) who is likely to be responsive to treatment with a combination of arimoclomol and miglustat, the method comprising: a) determining if the subject has an NPC genotype of: i) Missense/Missense; ii) Missense/Functional Null; or iii) Functional Null/Functional Null; and b) identifying the subject as being likely to respond to treatment with a combination of arimoclomol and miglustat when the subject is determined to have an NPC genotype of Missense/Missense or Missense/Functional Null or identifying the subject as less likely to respond to treatment with a combination arimoclomol and miglustat when the subject is determined to have an NPC genotype of Functional Null/Functional Null.

The present disclosure provides a method of treating or preventing Niemann Pick disease, type C (NPC) in a subject in need thereof, the method comprising: a) determining if the subject has an NPC genotype of: i) Missense/Missense; ii) Missense/Functional Null; or iii) Functional Null/Functional Null; and b) administering a therapeutically effective amount of arimoclomol to the subject when the subject is determined to have an NPC genotype of Missense/Missense or Missense/Functional Null.

The present disclosure also provides a method of treating or preventing Niemann Pick disease, type C (NPC) in a subject in need thereof, the method comprising: a) determining if the subject has an NPC genotype of: i) Missense/Missense; ii) Missense/Functional Null; or iii) Functional Null/Functional Null; and b) administering a therapeutically effective amount of arimoclomol and a therapeutically effective amount of miglustat to the subject when the subject is determined to have an NPC genotype of Missense/Missense or Missense/Functional Null.

In some embodiments of the preceding methods, the NPC genotype is an NPC1 genotype.

In some embodiments of the preceding methods the NPC genotype is an NPC2 genotype.

In some embodiments of the preceding methods, determining an NPC genotype (e.g. an NPC1 genotype and/or an NPC2 genotype) comprises sequencing the nucleic acid isolated from a biological sample from the subject. As would be appreciated by the skilled artisan, an NPC genotype (e.g.an NPC1 genotype and/or an NPC2 genotype) can be determined using any genotyping method known in the art.

The present disclosure provides a method of treating or preventing Niemann Pick disease, type C (NPC) in a subject in need thereof, the method comprising administering a therapeutically effective amount of arimoclomol to the subject, wherein the subject is identified as having at least one missense mutation in at least one of the two alleles in an NPC gene.

The present disclosure also provides a method of treating or preventing Niemann Pick disease, type C (NPC) in a subject in need thereof, the method comprising administering a therapeutically effective amount of arimoclomol and a therapeutically effective amount of miglustat to the subject, wherein the subject is identified as having at least one missense mutation in at least one of the two alleles in an NPC gene.

The present disclosure provides a method of predicting the responsiveness of a subject with Niemann Pick disease, type C (NPC) to treatment with arimoclomol, the method comprising: a) determining the presence or absence of at least one missense mutation in at least one allele of an NPC gene; and b) predicting that the subject will respond to treatment with arimoclomol when the presence of at least one missense mutation in at least one allele of an NPC gene is determined.

The present disclosure also provides a method of predicting the responsiveness of a subject with Niemann Pick disease, type C (NPC) to treatment with a combination of arimoclomol and miglustat, the method comprising: a) determining the presence or absence of at least one missense mutation in at least one allele of an NPC gene; and b) predicting that the subject will respond to treatment with a combination of arimoclomol and miglustat when the presence of at least one missense mutation in at least one allele of an NPC gene is determined.

The present disclosure provides a method of identifying a subject with Niemann Pick disease, type C (NPC) who will be responsive to treatment with arimoclomol, the method comprising: a) determining the presence or absence of at least one missense mutation in at least one allele of an NPC gene; and b) identifying that the subject will be responsive to treatment with arimoclomol when the presence of at least one missense mutation in at least one allele of an NPC gene is determined.

The present disclosure provides a method of identifying a subject with Niemann Pick disease, type C (NPC) who will be responsive to treatment with a combination of arimoclomol and miglustat, the method comprising: a) determining the presence or absence of at least one missense mutation in at least one allele of an NPC gene; and b) identifying that the subject will be responsive to treatment with a combination of arimoclomol and miglustat when the presence of at least one missense mutation in at least one allele of an NPC gene is determined.

The present disclosure provides a method of treating or preventing Niemann Pick disease, type C (NPC) in a subject in need thereof, the method comprising: a) determining the presence or absence of at least one missense mutation in at least one allele of an NPC gene; and b) administering a therapeutically effective amount of arimoclomol to the subject when the presence of at least one missense mutation in at least one allele of an NPC gene is determined.

The present disclosure provides a method of treating or preventing Niemann Pick disease, type C (NPC) in a subject in need thereof, the method comprising: a) determining the presence or absence of at least one missense mutation in at least one allele of an NPC gene; and b) administering a therapeutically effective amount of arimoclomol and a therapeutically effective amount of miglustat to the subject when the presence of at least one missense mutation in at least one allele of an NPC gene is determined.

In some embodiments of the preceding methods, the subject is identified as having at least one missense mutation in each of the two alleles of the NPC gene.

In some embodiments of the preceding methods, the subject is identified as having at least one missense mutation in one allele of the NPC gene and a functional null mutation in the other allele of the NPC gene.

In some embodiments of the preceding methods, the subject is identified as having a functional null mutation in at most one allele of the two alleles of the NPC gene.

In some embodiments of the preceding methods, the subject is identified as being a compound heterozygote for the NPC gene.

In some embodiments of the preceding methods, the NPC gene is NPC1.

In some embodiments of the preceding methods, the NPC gene is NPC2.

In some embodiments of the preceding methods, determining the presence or absence of at least one missense mutation in at least one allele of the NPC gene comprises sequencing nucleic acid isolated from a biological sample from the subject. As would be appreciated by the skilled artisan, a mutation in an NPC gene (e.g. a mutation in NPC1 or a mutation in NPC2) can be determined using any method known in the art for determining the mutational status of a gene within the genome of a subject.

In some embodiments of the preceding methods, the functional null mutation is a frameshift mutation. In some embodiments, the frameshift mutation is selected from Q991fs, D508fs, H641fs, A1108fs, L1204fs, Q119fs, P733fs, L773fs, K822fs, I962fs, A1192fs, and L1248fs. In some embodiments, the functional null mutation is Q991fs.

In some embodiments of the preceding methods, the functional null mutation is an aberrant splicing mutation. In some embodiments, the aberrant splicing mutation is selected from R518Q/sp, V2Osp, G319sp, F1199sp1, and F1199sp2.

In some embodiments of the preceding methods, the functional null mutation is a premature stop codon mutation. In some embodiments, the premature stop codon mutation is selected from R116*, L860*, R934*, and E1188*.

In some embodiments of the preceding methods, the missense mutation results in a single amino-acid change. In some embodiments of the preceding methods, the missense mutation results in a substitution, a deletion, an insertion, a duplication, or any combination thereof

In some embodiments, the missense mutation is selected from I1061T, P1007A, S940L, R1186H, G886V, R978C, P1007L, L472P, R518W, G910S, A927V, S954L, G992W, S1004P, T1036M, N1156S, V1165M, N222S, G248V, S357L, R404W, E451K, Y677N, E718D, S734I, G765V, H897Q, A926V, N968S, P981L, M1001V, P1007R, H1016L, T1036A, A1054T, R1059Q, V1078I, F1079L, F1087L, V1141G, M1142T, A1151T, V1155G, and T1205K. In some embodiments, the missense mutation is selected from 11061T, P1007A, M1142T, N1156S, R1186H, S734I, S940L, and S954L. In some embodiments, the missense mutation is selected from I1061T and P1007A. In some embodiments, the missense mutation is selected from R978C and N222S.

In some embodiments, the missense mutation is selected from G886V, Y677N, E718D, G765V, H897Q, V1078I, V1155G.

In some embodiments, the functional null mutation is selected from D508fs, L773fs, K822fs, I962fs, F1199sp1, F1199sp2.

In some embodiments, the functional null mutation is selected from D508fs, L773fs, K822fs, I962fs.

In some embodiments, the subject has an NPC1 genotype selected from the group consisting of G886V/R978C, Y677N/R1059Q, E718D/P1007L, G765V/P1007A, A1054T/H897Q, P733fs/V10781, T1036A/V1155G, D508fs/I1061T, G248V/L773fs, S734I/K822fs, I1061T/I962fs, V1165M/F1199sp1, and N1156S/F1199sp2.

In some embodiments, the subject has an NPC1 genotype selected from the group consisting of G886V/R978C, Y677N/R1059Q, E718D/P1007L, G765V/P1007A, A1054T/H897Q, P733fs/V10781, T1036A/V1155G, D508fs/I1061T, G248V/L773fs, S734I/K822fs, and I1061T/I962fs.

In some embodiments of the preceding methods, the missense mutation is an endoplasmic reticulum (ER) type missense mutation. In some embodiments, the ER type missense mutation is selected from the group consisting of I1061T, M1142T, N1156S and R1186H.

In some embodiments of the preceding methods, the subject has an NPC1 genotype selected from the group consisting of 11061T/E1188*, I1061T/A1151T, I1061T/Q119fs, I1061T/I962fs, T1036M/11061T, I1061T/V1141G, N968S/R1186H, N1156S/F1199sp2, Q991fs/I1061T, H1016L/I1061T, I1061T/A1192fs, I1061T/N1156S, R1186H/R1186H, P1007A/R1186H, and I1061T/D508fs.

In some embodiments of the preceding methods, the subject has an NPC1 genotype selected from the NPC1 genotypes recited in Table 1.

TABLE 1
NPC1 genotypes

| NPC1 Allele 1 | NPC1 Allele 2 | |
|---|---|---|
| Amino Acid | Amino Acid | NPC1 Genotype |
| V20sp | P1007A | Missense/Functional Null |
| Q991fs | P1007A | Missense/Functional Null |
| A927V | S1004P | Missense/Missense |
| S940L | P1007A | Missense/Missense |
| I1061T | E1188* | Missense/Functional Null |
| A926V | P1007A | Missense/Missense |
| R404W | M1001V | Missense/Missense |
| I1061T | A1151T | Missense/Missense |
| I1061T | Q119fs | Missense/Functional Null |
| I1061T | I962fs | Missense/Functional Null |
| S734I | K822fs | Missense/Functional Null |
| P1007L | P1007L | Missense/Missense |
| T1036M | I1061T | Missense/Missense |
| G765V | P1007A | Missense/Missense |
| I1061T | N1156S | Missense/Missense |
| I1061T | V1141G | Missense/Missense |
| E718D | P1007L | Missense/Missense |
| V1165M | R116* | Missense/Functional Null |
| R1186H | R1186H | Missense/Missense |
| N968S | R1186H | Missense/Missense |
| E451K | G992W | Missense/Missense |
| P1007R | T1205K | Missense/Missense |
| R934* | F1079L | Missense/Functional Null |
| N1156S | F1199sp2 | Missense/Functional Null |
| V1165M | F1199sp1 | Missense/Functional Null |
| R518W | H641fs | Missense/Functional Null |
| G248V | L773fs | Missense/Functional Null |
| P1007A | R1186H | Missense/Missense |
| A1054T | H897Q | Missense/Missense |
| P733fs | V1078I | Missense/Functional Null |
| H641fs | S954L | Missense/Functional Null |
| D508fs | I1061T | Missense/Functional Null |
| Q991fs | I1061T | Missense/Functional Null |
| G910S | G910S | Missense/Missense |
| P1007A | L1204fs | Missense/Functional Null |
| P1007A | L1204fs | Missense/Functional Null |
| G886V | R978C | Missense/Missense |
| G886V | R978C | Missense/Missense |
| S357L | S940L | Missense/Missense |
| R518W | G992W | Missense/Missense |
| S940L | S940L | Missense/Missense |
| Y677N | R1059Q | Missense/Missense |
| T1036A | V1155G | Missense/Missense |
| H1016L | I1061T | Missense/Missense |
| P981L | L1248fs | Missense/Functional Null |
| L472P | L472P | Missense/Missense |
| I1061T | A1192fs | Missense/Functional Null |

In some embodiments, arimoclomol is administered orally. In some embodiments, arimoclomol is administered via a feeding tube.

In some embodiments, arimoclomol administration provides sustained benefit over a period of time.

In some embodiments, the period of time is a six-month period. In some embodiments, the period of time is a one-year period. In some embodiments, the period of time is a one-year and six-month period. In some embodiments, the period of time is a two-year period. In some embodiments, the period of time is a two-year and six-month period. In some embodiments, the period of time is a three-year period. In some embodiments, the period of time is a three-year and six month period. In some embodiments, the period of time is a four year period. In some embodiments, the period of time is a four-year and six month period. In some embodiments, the period of time is a five year period.

As would be appreciated by the skilled artisan, progression of NPC in a subject can be tracked by a clinician using the NPC composite clinical severity scale (hereafter “NPCCSS”; see Yanjanin NM et al. Am J Med Genet B Neuropsychiatr Genet, 2010; 153B (1): 132-140, incorporated herein by reference in its entirety). A full “17-domain NPCCSS score” incorporates clinical signs and symptoms in nine major (ambulation, cognition, eye movement, fine motor, hearing, memory, seizures, speech, swallowing) and eight minor (auditory brainstem response, behavior, gelastic cataplexy, hyperreflexia, incontinence, narcolepsy, psychiatric, respiratory problems) domains to determine a score which describes the severity of the subject's NPC progression (a higher score, the more progressed/severe the disease is). An abridged “5-domain NPCCSS score” is also used by clinicians, and incorporates clinical signs and symptoms from the major domains of ambulation, cognition, fine motor, speech and swallowing (see Cortina-Borj a M et al. OrphanetJRare Dis, 2018; 13 (1): 143; 3, incorporated herein by reference in its entirety).

As would be appreciated by the skilled artisan, subgroups of subjects with a patient population of NPC can also be analysed using an annual severity increment score (hereafter “ASIS”) which is calculated by dividing the total NPCCSS score by the age of the subject, thereby providing a measure of the rate of disease progression in an individual subject. Accordingly, the resulting value serves as an index of the annual rate of disease progression. See Cortina-Borja M et al. Orphanet J Rare Dis, 2018; 13 (1): 143; 3, incorporated herein by reference in its entirety.

The present disclosure provides a method of treating or preventing Niemann Pick disease, type C (NPC) in a subject in need thereof, the method comprising administering a therapeutically effective amount of arimoclomol to the subject, wherein the subject has an ASIS between about 0.5 and about 2.

The present disclosure also provides a method of treating or preventing Niemann Pick disease, type C (NPC) in a subject in need thereof, the method comprising administering a therapeutically effective amount of arimoclomol and a therapeutically effective amount of miglustat to the subject, wherein the subject has an ASIS between about 0.5 and about 2.

In some embodiments, arimoclomol is administered for about six-months or more. In some embodiments, arimoclomol is administered for about one-year or more.

In some embodiments, arimoclomol is administered for about a six-month period. In some embodiments, arimoclomol is administered for about a one-year period. In some embodiments, arimoclomol is administered for about a one-year and six-month period. In some embodiments, arimoclomol is administered for greater than a one-year period. In some embodiments, arimoclomol is administered for about a two-year period. In some embodiments, arimoclomol is administered for about a two-year and six-month period. In some embodiments, arimoclomol is administered for greater than a two-year period. In some embodiments, arimoclomol is administered for about a three-year period. In some embodiments, arimoclomol is administered for greater than a three-year period.

In some embodiments, arimoclomol is administered as the citrate salt (e.g., arimoclomol citrate).

In some embodiments, the disease course is modified.

In some embodiments, the subject is a mammal. In some embodiments, the mammal is a human.

In some embodiments, the subject is between the ages of about 0 years old to about 5 years old. In some embodiments, the subject is between the ages of about 5 years old to about 10 years old. In some embodiments, the subject is between the ages of about 10 years old to about 15 years old. In some embodiments, the subject is between the ages of about 15 years old to about 20 years old. In some embodiments, the subject is between the ages of about 20 years old to about 30 years old. In some embodiments, the subject is between the ages of about 30 years old to about 40 years old. In some embodiments, the subject is between the ages of about 40 years old to about 50 years old. In some embodiments, the subject is between the ages of about 50 years old to about 60 years old. In some embodiments, the subject is between the ages of about 60 years old to about 70 years old. In some embodiments, the subject is between the ages of about 70 years old to about 80 years old. In some embodiments, the subject is between the ages of about 80 years old to about 90 years old. In some embodiments, the subject is between the ages of about 90 years old to about 100 years old. In some embodiments, the subject is between the ages of about 100 years old to about 110 years old.

In some embodiments, the subject is about 1 year old. In some embodiments, the subject is about 2 years old. In some embodiments, the subject is about 3 years old. In some embodiments, the subject is about 4 years old. In some embodiments, the subject is about 5 years old. In some embodiments, the subject is about 6 years old. In some embodiments, the subject is about 7 years old. In some embodiments, the subject is about 8 years old. In some embodiments, the subject is about 9 years old. In some embodiments, the subject is about 10 years old. In some embodiments, the subject is about 11 years old. In some embodiments, the subject is about 12 years old. In some embodiments, the subject is about 13 years old. In some embodiments, the subject is about 14 years old. In some embodiments, the subject is about 15 years old. In some embodiments, the subject is about 16 years old. In some embodiments, the subject is about 17 years old. In some embodiments, the subject is about 18 years old. In some embodiments, the subject is about 19 years old. In some embodiments, the subject is about 20 years old. In some embodiments, the subject is about 21 years old. In some embodiments, the subject is about 22 years old. In some embodiments, the subject is about 23 years old. In some embodiments, the subject is about 24 years old. In some embodiments, the subject is about 25 years old. In some embodiments, the subject is about 26 years old. In some embodiments, the subject is about 27 years old. In some embodiments, the subject is about 28 years old. In some embodiments, the subject is about 29 years old. In some embodiments, the subject is about 30 years old.

In some embodiments, arimoclomol citrate is administered from about 100 mg/day to about 1000 mg/day. In some embodiments, arimoclomol citrate is administered from about 150 mg/day to about 1000 mg/day. In some embodiments, arimoclomol citrate is administered from about 200 mg/day to about 1000 mg/day. In some embodiments, arimoclomol citrate is administered from about 250 mg/day to about 1000 mg/day. In some embodiments, arimoclomol citrate is administered from about 300 mg/day to about 1000 mg/day. In some embodiments, arimoclomol citrate is administered from about 350 mg/day to about 1000 mg/day. In some embodiments, arimoclomol citrate is administered from about 400 mg/day to about 1000 mg/day. In some embodiments, arimoclomol citrate is administered from about 450 mg/day to about 1000 mg/day. In some embodiments, arimoclomol citrate is administered from about 500 mg/day to about 1000 mg/day. In some embodiments, arimoclomol citrate is administered from about 550 mg/day to about 1000 mg/day. In some embodiments, arimoclomol citrate is administered from about 600 mg/day to about 1000 mg/day. In some embodiments, arimoclomol citrate is administered from about 650 mg/day to about 1000 mg/day. In some embodiments, arimoclomol citrate is administered from about 700 mg/day to about 1000 mg/day. In some embodiments, arimoclomol citrate is administered from about 750 mg/day to about 1000 mg/day. In some embodiments, arimoclomol citrate is administered from about 800 mg/day to about 1000 mg/day. In some embodiments, arimoclomol citrate is administered from about 850 mg/day to about 1000 mg/day. In some embodiments, arimoclomol citrate is administered from about 900 mg/day to about 1000 mg/day. In some embodiments, arimoclomol citrate is administered from about 950 mg/day to about 1000 mg/day.

In some embodiments, arimoclomol citrate is administered from about 100 mg/day to about 950 mg/day. In some embodiments, arimoclomol citrate is administered from about 100 mg/day to about 900 mg/day. In some embodiments, arimoclomol citrate is administered from about 100 mg/day to about 850 mg/day. In some embodiments, arimoclomol citrate is administered from about 100 mg/day to about 800 mg/day. In some embodiments, arimoclomol citrate is administered from about 100 mg/day to about 750 mg/day. In some embodiments, arimoclomol citrate is administered from about 100 mg/day to about 700 mg/day. In some embodiments, arimoclomol citrate is administered from about 100 mg/day to about 650 mg/day. In some embodiments, arimoclomol citrate is administered from about 100 mg/day to about 600 mg/day. In some embodiments, arimoclomol citrate is administered from about 100 mg/day to about 550 mg/day. In some embodiments, arimoclomol citrate is administered from about 100 mg/day to about 500 mg/day. In some embodiments, arimoclomol citrate is administered from about 100 mg/day to about 450 mg/day. In some embodiments, arimoclomol citrate is administered from about 100 mg/day to about 400 mg/day. In some embodiments, arimoclomol citrate is administered from about 100 mg/day to about 350 mg/day. In some embodiments, arimoclomol citrate is administered from about 100 mg/day to about 300 mg/day. In some embodiments, arimoclomol citrate is administered from about 100 mg/day to about 250 mg/day. In some embodiments, arimoclomol citrate is administered from about 100 mg/day to about 200 mg/day. In some embodiments, arimoclomol citrate is administered from about 100 mg/day to about 150 mg/day.

In some embodiments, arimoclomol citrate is administered at about 100 mg/day, about 125 mg/day, about 150 mg/day, about 175 mg/day, about 200 mg/day, about 225 mg/day, about 250 mg/day, about 275 mg/day, about 300 mg/day, about 325 mg/day, about 350 mg/day, about 375 mg/day, about 400 mg/day, about 425 mg/day, about 450 mg/day, about 475 mg/day, about 500 mg/day, about 525 mg/day, about 550 mg/day, about 575 mg/day, about 600 mg/day, about 625 mg/day, about 650 mg/day, about 675 mg/day, or about 700 mg/day.

In some embodiments, arimoclomol citrate is administered at about 100 mg/day. In some embodiments, arimoclomol citrate is administered at about 125 mg/day. In some embodiments, arimoclomol citrate is administered at about 150 mg/day. In some embodiments, arimoclomol citrate is administered at about 175 mg/day. In some embodiments, arimoclomol citrate is administered at about 200 mg/day. In some embodiments, arimoclomol citrate is administered at about 225 mg/day. In some embodiments, arimoclomol citrate is administered at about 250 mg/day. In some embodiments, arimoclomol citrate is administered at about 275 mg/day. In some embodiments, arimoclomol citrate is administered at about 300 mg/day. In some embodiments, arimoclomol citrate is administered at about 325 mg/day. In some embodiments, arimoclomol citrate is administered at about 350 mg/day. In some embodiments, arimoclomol citrate is administered at about 375 mg/day. In some embodiments, arimoclomol citrate is administered at about 400 mg/day. In some embodiments, arimoclomol citrate is administered at about 425 mg/day. In some embodiments, arimoclomol citrate is administered at about 450 mg/day. In some embodiments, arimoclomol citrate is administered at about 475 mg/day. In some embodiments, arimoclomol citrate is administered at about 500 mg/day. In some embodiments, arimoclomol citrate is administered at about 525 mg/day. In some embodiments, arimoclomol citrate is administered at about 550 mg/day. In some embodiments, arimoclomol citrate is administered at about 575 mg/day. In some embodiments, arimoclomol citrate is administered at about 600 mg/day. In some embodiments, arimoclomol citrate is administered at about 625 mg/day. In some embodiments, arimoclomol citrate is administered at about 650 mg/day. In some embodiments, arimoclomol citrate is administered at about 675 mg/day. In some embodiments, arimoclomol citrate is administered at about 700 mg/day.

In some embodiments, arimoclomol citrate is administered at about 25 mg to about 300 mg. In some embodiments, arimoclomol citrate is administered at about 50 mg to about 300 mg. In some embodiments, arimoclomol citrate is administered at about 75 mg to about 300 mg. In some embodiments, arimoclomol citrate is administered at about 100 mg to about 300 mg. In some embodiments, arimoclomol citrate is administered at about 125 mg to about 300 mg. In some embodiments, arimoclomol citrate is administered at about 150 mg to about 300 mg. In some embodiments, arimoclomol citrate is administered at about 175 mg to about 300 mg. In some embodiments, arimoclomol citrate is administered at about 200 mg to about 300 mg. In some embodiments, arimoclomol citrate is administered at about 225 mg to about 300 mg. In some embodiments, arimoclomol citrate is administered at about 250 mg to about 300 mg. In some embodiments, arimoclomol citrate is administered at about 275 mg to about 300 mg.

In some embodiments, arimoclomol citrate is administered at about 25 mg to about 275 mg. In some embodiments, arimoclomol citrate is administered at about 25 mg to about 250 mg. In some embodiments, arimoclomol citrate is administered at about 25 mg to about 225 mg. In some embodiments, arimoclomol citrate is administered at about 25 mg to about 200 mg. In some embodiments, arimoclomol citrate is administered at about 25 mg to about 175 mg. In some embodiments, arimoclomol citrate is administered at about 25 mg to about 150 mg. In some embodiments, arimoclomol citrate is administered at about 25 mg to about 125 mg. In some embodiments, arimoclomol citrate is administered at about 25 mg to about 100 mg. In some embodiments, arimoclomol citrate is administered at about 25 mg to about 75 mg. In some embodiments, arimoclomol citrate is administered at about 25 mg to about 50 mg.

In some embodiments, arimoclomol citrate is administered at about 25 mg. In some embodiments, arimoclomol citrate is administered at about 50 mg. In some embodiments, arimoclomol citrate is administered at about 75 mg. In some embodiments, arimoclomol citrate is administered at about 100 mg. In some embodiments, arimoclomol citrate is administered at about 125 mg. In some embodiments, arimoclomol citrate is administered at about 150 mg. In some embodiments, arimoclomol citrate is administered at about 175 mg. In some embodiments, arimoclomol citrate is administered at about 200 mg. In some embodiments, arimoclomol citrate is administered at about 225 mg. In some embodiments, arimoclomol citrate is administered at about 250 mg. In some embodiments, arimoclomol citrate is administered at about 275 mg. In some embodiments, arimoclomol citrate is administered at about 300 mg.

In some embodiments, arimoclomol is administered from about 53 mg/day to about 423 mg/day. In some embodiments, arimoclomol is administered from about 63 mg/day to about 423 mg/day. In some embodiments, arimoclomol is administered from about 73 mg/day to about 423 mg/day. In some embodiments, arimoclomol is administered from about 83 mg/day to about 423 mg/day. In some embodiments, arimoclomol is administered from about 93 mg/day to about 423 mg/day. In some embodiments, arimoclomol is administered from about 103 mg/day to about 423 mg/day. In some embodiments, arimoclomol is administered from about 113 mg/day to about 423 mg/day. In some embodiments, arimoclomol is administered from about 123 mg/day to about 423 mg/day. In some embodiments, arimoclomol is administered from about 133 mg/day to about 423 mg/day. In some embodiments, arimoclomol is administered from about 143 mg/day to about 423 mg/day. In some embodiments, arimoclomol is administered from about 153 mg/day to about 423 mg/day. In some embodiments, arimoclomol is administered from about 163 mg/day to about 423 mg/day. In some embodiments, arimoclomol is administered from about 173 mg/day to about 423 mg/day. In some embodiments, arimoclomol is administered from about 183 mg/day to about 423 mg/day. In some embodiments, arimoclomol is administered from about 193 mg/day to about 423 mg/day. In some embodiments, arimoclomol is administered from about 203 mg/day to about 423 mg/day. In some embodiments, arimoclomol is administered from about 213 mg/day to about 423 mg/day. In some embodiments, arimoclomol is administered from about 223 mg/day to about 423 mg/day. In some embodiments, arimoclomol is administered from about 243 mg/day to about 423 mg/day. In some embodiments, arimoclomol is administered from about 253 mg/day to about 423 mg/day. In some embodiments, arimoclomol is administered from about 263 mg/day to about 423 mg/day. In some embodiments, arimoclomol is administered from about 273 mg/day to about 423 mg/day. In some embodiments, arimoclomol is administered from about 283 mg/day to about 423 mg/day. In some embodiments, arimoclomol is administered from about 293 mg/day to about 423 mg/day. In some embodiments, arimoclomol is administered from about 303 mg/day to about 423 mg/day. In some embodiments, arimoclomol is administered from about 313 mg/day to about 423 mg/day. In some embodiments, arimoclomol is administered from about 323 mg/day to about 423 mg/day. In some embodiments, arimoclomol is administered from about 333 mg/day to about 423 mg/day. In some embodiments, arimoclomol is administered from about 343 mg/day to about 423 mg/day. In some embodiments, arimoclomol is administered from about 353 mg/day to about 423 mg/day. In some embodiments, arimoclomol is administered from about 363 mg/day to about 423 mg/day. In some embodiments, arimoclomol is administered from about 373 mg/day to about 423 mg/day. In some embodiments, arimoclomol is administered from about 383 mg/day to about 423 mg/day. In some embodiments, arimoclomol is administered from about 393 mg/day to about 423 mg/day. In some embodiments, arimoclomol is administered from about 403 mg/day to about 423 mg/day. In some embodiments, arimoclomol is administered from about 413 mg/day to about 423 mg/day.

In some embodiments, arimoclomol is administered from about 53 mg/day to about 413 mg/day. In some embodiments, arimoclomol is administered from about 53 mg/day to about 403 mg/day. In some embodiments, arimoclomol is administered from about 53 mg/day to about 393 mg/day. In some embodiments, arimoclomol is administered from about 53 mg/day to about 383 mg/day. In some embodiments, arimoclomol is administered from about 53 mg/day to about 373 mg/day. In some embodiments, arimoclomol is administered from about 53 mg/day to about 363 mg/day. In some embodiments, arimoclomol is administered from about 53 mg/day to about 353 mg/day. In some embodiments, arimoclomol is administered from about 53 mg/day to about 343 mg/day. In some embodiments, arimoclomol is administered from about 53 mg/day to about 333 mg/day. In some embodiments, arimoclomol is administered from about 53 mg/day to about 323 mg/day. In some embodiments, arimoclomol is administered from about 53 mg/day to about 313 mg/day. In some embodiments, arimoclomol is administered from about 53 mg/day to about 303 mg/day. In some embodiments, arimoclomol is administered from about 53 mg/day to about 293 mg/day. In some embodiments, arimoclomol is administered from about 53 mg/day to about 283 mg/day. In some embodiments, arimoclomol is administered from about 53 mg/day to about 273 mg/day. In some embodiments, arimoclomol is administered from about 53 mg/day to about 263 mg/day. In some embodiments, arimoclomol is administered from about 53 mg/day to about 253 mg/day. In some embodiments, arimoclomol is administered from about 53 mg/day to about 243 mg/day. In some embodiments, arimoclomol is administered from about 53 mg/day to about 233 mg/day. In some embodiments, arimoclomol is administered from about 53 mg/day to about 223 mg/day. In some embodiments, arimoclomol is administered from about 53 mg/day to about 213 mg/day. In some embodiments, arimoclomol is administered from about 53 mg/day to about 203 mg/day. In some embodiments, arimoclomol is administered from about 53 mg/day to about 193 mg/day. In some embodiments, arimoclomol is administered from about 53 mg/day to about 183 mg/day. In some embodiments, arimoclomol is administered from about 53 mg/day to about 173 mg/day. In some embodiments, arimoclomol is administered from about 53 mg/day to about 163 mg/day. In some embodiments, arimoclomol is administered from about 53 mg/day to about 153 mg/day. In some embodiments, arimoclomol is administered from about 53 mg/day to about 143 mg/day. In some embodiments, arimoclomol is administered from about 53 mg/day to about 133 mg/day. In some embodiments, arimoclomol is administered from about 53 mg/day to about 123 mg/day. In some embodiments, arimoclomol is administered from about 53 mg/day to about 113 mg/day. In some embodiments, arimoclomol is administered from about 53 mg/day to about 103 mg/day. In some embodiments, arimoclomol is administered from about 53 mg/day to about 93 mg/day. In some embodiments, arimoclomol is administered from about 53 mg/day to about 83 mg/day. In some embodiments, arimoclomol is administered from about 53 mg/day to about 73 mg/day. In some embodiments, arimoclomol is administered from about 53 mg/day to about 63 mg/day.

In some embodiments, arimoclomol is administered at about 53 mg/day, about 63 mg/day, about 73 mg/day, about 83 mg/day, about 93 mg/day, about 103 mg/day, about 113 mg/day, about 123 mg/day, about 133 mg/day, about 134 mg/day, about 143 mg/day, about 153 mg/day, about 163 mg/day, about 173 mg/day, about 183 mg/day, about 193 mg/day, about 203 mg/day, about 213 mg/day, about 223 mg/day, about 233 mg/day, about 243 mg/day, about 253 mg/day, about 263 mg/day, about 273 mg/day, about 283 mg/day, about 293 mg/day, about 303 mg/day, about 313 mg/day, about 323 mg/day, about 333 mg/day, about 343 mg/day, about 353 mg/day, about 363 mg/day, about 373 mg/day, about 383 mg/day, about 393 mg/day, about 403 mg/day, about 413 mg/day, or about 423 mg/day.

In some embodiments, arimoclomol is administered at about 53 mg/day. In some embodiments, arimoclomol is administered at about 63 mg/day. In some embodiments, arimoclomol is administered at about 73 mg/day. In some embodiments, arimoclomol is administered at about 83 mg/day. In some embodiments, arimoclomol is administered at about 93 mg/day. In some embodiments, arimoclomol is administered at about 103 mg/day. In some embodiments, arimoclomol is administered at about 113 mg/day. In some embodiments, arimoclomol is administered at about 123 mg/day. In some embodiments, arimoclomol is administered at about 133 mg/day. In some embodiments, arimoclomol is administered at about 134 mg/day. In some embodiments, arimoclomol is administered at about 143 mg/day. In some embodiments, arimoclomol is administered at about 153 mg/day. In some embodiments, arimoclomol is administered at about 163 mg/day. In some embodiments, arimoclomol is administered at about 173 mg/day. In some embodiments, arimoclomol is administered at about 183 mg/day. In some embodiments, arimoclomol is administered at about 193 mg/day. In some embodiments, arimoclomol is administered at about 203 mg/day. In some embodiments, arimoclomol is administered at about 213 mg/day. In some embodiments, arimoclomol is administered at about 223 mg/day. In some embodiments, arimoclomol is administered at about 233 mg/day. In some embodiments, arimoclomol is administered at about 243 mg/day. In some embodiments, arimoclomol is administered at about 253 mg/day. In some embodiments, arimoclomol is administered at about 263 mg/day. In some embodiments, arimoclomol is administered at about 273 mg/day. In some embodiments, arimoclomol is administered at about 283 mg/day. In some embodiments, arimoclomol is administered at about 293 mg/day. In some embodiments, arimoclomol is administered at about 303 mg/day. In some embodiments, arimoclomol is administered at about 313 mg/day. In some embodiments, arimoclomol is administered at about 323 mg/day. In some embodiments, arimoclomol is administered at about 333 mg/day. In some embodiments, arimoclomol is administered at about 343 mg/day. In some embodiments, arimoclomol is administered at about 353 mg/day. In some embodiments, arimoclomol is administered at about 363 mg/day. In some embodiments, arimoclomol is administered at about 373 mg/day. In some embodiments, arimoclomol is administered at about 383 mg/day. In some embodiments, arimoclomol is administered at about 393 mg/day. In some embodiments, arimoclomol is administered at about 403 mg/day. In some embodiments, arimoclomol is administered at about 413 mg/day. In some embodiments, arimoclomol is administered at about 423 mg/day.

In some embodiments, arimoclomol is administered at about 372 mg/day.

In some embodiments, arimoclomol is administered at about 13 mg to about 143 mg. In some embodiments, arimoclomol is administered at about 23 mg to about 143 mg. In some embodiments, arimoclomol is administered at about 33 mg to about 143 mg. In some embodiments, arimoclomol is administered at about 43 mg to about 143 mg. In some embodiments, arimoclomol is administered at about 53 mg to about 143 mg. In some embodiments, arimoclomol is administered at about 63 mg to about 143 mg. In some embodiments, arimoclomol is administered at about 73 mg to about 143 mg. In some embodiments, arimoclomol is administered at about 83 mg to about 143 mg. In some embodiments, arimoclomol is administered at about 93 mg to about 143 mg. In some embodiments, arimoclomol is administered at about 103 mg to about 143 mg. In some embodiments, arimoclomol is administered at about 113 mg to about 143 mg. In some embodiments, arimoclomol is administered at about 123 mg to about 143 mg. In some embodiments, arimoclomol is administered at about 133 mg to about 143 mg.

In some embodiments, arimoclomol is administered at about 13 mg to about 133 mg. In some embodiments, arimoclomol is administered at about 13 mg to about 123 mg. In some embodiments, arimoclomol is administered at about 13 mg to about 113 mg. In some embodiments, arimoclomol is administered at about 13 mg to about 103 mg. In some embodiments, arimoclomol is administered at about 13 mg to about 93 mg. In some embodiments, arimoclomol is administered at about 13 mg to about 83 mg. In some embodiments, arimoclomol is administered at about 13 mg to about 73 mg. In some embodiments, arimoclomol is administered at about 13 mg to about 63 mg. In some embodiments, arimoclomol is administered at about 13 mg to about 53 mg. In some embodiments, arimoclomol is administered at about 13 mg to about 43 mg. In some embodiments, arimoclomol is administered at about 13 mg to about 33 mg. In some embodiments, arimoclomol is administered at about 13 mg to about 23 mg.

In some embodiments, arimoclomol is administered at about 13 mg. In some embodiments, arimoclomol is administered at about 23 mg. In some embodiments, arimoclomol is administered at about 33 mg. In some embodiments, arimoclomol is administered at about 43 mg. In some embodiments, arimoclomol is administered at about 53 mg. In some embodiments, arimoclomol is administered at about 63 mg. In some embodiments, arimoclomol is administered at about 73 mg. In some embodiments, arimoclomol is administered at about 83 mg. In some embodiments, arimoclomol is administered at about 93 mg. In some embodiments, arimoclomol is administered at about 103 mg. In some embodiments, arimoclomol is administered at about 113 mg. In some embodiments, arimoclomol is administered at about 123 mg. In some embodiments, arimoclomol is administered at about 133 mg. In some embodiments, arimoclomol is administered at about 143 mg.

In some embodiments, arimoclomol is administered at about 124 mg.

In some embodiments, arimoclomol is administered one time, two times, three times, four times, or five times daily.

In some embodiments, arimoclomol citrate is administered one time, two times, three times, four times, or five times daily.

In some embodiments, arimoclomol is administered one time daily. In some embodiments, arimoclomol is administered two times daily. In some embodiments, arimoclomol is administered three times daily. In some embodiments, arimoclomol is administered four times daily. In some embodiments, arimoclomol is administered five times daily.

In some embodiments, arimoclomol citrate is administered one time daily. In some embodiments, arimoclomol citrate is administered two times daily. In some embodiments, arimoclomol citrate is administered three times daily. In some embodiments, arimoclomol citrate is administered four times daily. In some embodiments, arimoclomol citrate is administered five times daily.

In some embodiments, arimoclomol citrate is administered three times daily at about 93 mg/day to about 372 mg/day.

In some embodiments, arimoclomol is administered one, two, three, four, five, six, or seven days a week.

In some embodiments, arimoclomol citrate is administered one, two, three, four, five, six, or seven days a week.

In some embodiments, arimoclomol is administered one day a week. In some embodiments, arimoclomol is administered two days a week. In some embodiments, arimoclomol is administered three days a week. In some embodiments, arimoclomol is administered four days a week. In some embodiments, arimoclomol is administered five days a week. In some embodiments, arimoclomol is administered six days a week. In some embodiments, arimoclomol is administered seven days a week.

In some embodiments, arimoclomol citrate is administered one day a week. In some embodiments, arimoclomol citrate is administered two days a week. In some embodiments, arimoclomol citrate is administered three days a week. In some embodiments, arimoclomol citrate is administered four days a week. In some embodiments, arimoclomol citrate is administered five days a week. In some embodiments, arimoclomol citrate is administered six days a week. In some embodiments, arimoclomol citrate is administered seven days a week.

In some embodiments, arimoclomol is administered in a dosage adjusted by patient body weight.

In some embodiments, arimoclomol citrate is administered in a dosage adjusted by patient body weight.

In some embodiments, the subject has a body weight of about 8 kg to about 15 kg and arimoclomol citrate is administered at a dose of about 50 mg t.i.d. (i.e., about 150 mg/day).

In some embodiments, the subject has a body weight of about 8 kg and arimoclomol citrate is administered at a dose of about 50 mg t.i.d. (i.e., about 150 mg/day). In some embodiments, the subject has a body weight of about 9 kg and arimoclomol citrate is administered at a dose of about 50 mg t.i.d. (i.e., about 150 mg/day). In some embodiments, the subject has a body weight of about 10 kg and arimoclomol citrate is administered at a dose of about 50 mg t.i.d. (i.e., about 150 mg/day). In some embodiments, the subject has a body weight of about 11 kg and arimoclomol citrate is administered at a dose of about 50 mg t.i.d. (i.e., about 150 mg/day). In some embodiments, the subject has a body weight of about 12 kg and arimoclomol citrate is administered at a dose of about 50 mg t.i.d. (i.e., about 150 mg/day). In some embodiments, the subject has a body weight of about 13 kg and arimoclomol citrate is administered at a dose of about 50 mg t.i.d. (i.e., about 150 mg/day). In some embodiments, the subject has a body weight of about 14 kg and arimoclomol citrate is administered at a dose of about 50 mg t.i.d. (i.e., about 150 mg/day). In some embodiments, the subject has a body weight of about 15 kg and arimoclomol citrate is administered at a dose of about 50 mg t.i.d. (i.e., about 150 mg/day).

In some embodiments, the subject has a body weight of about 15 kg to about 22 kg and arimoclomol citrate is administered at a dose of about 75 mg t.i.d. (i.e., about 225 mg/day).

In some embodiments, the subject has a body weight of about 15 kg and arimoclomol citrate is administered at a dose of about 75 mg t.i.d. (i.e., about 225 mg/day). In some embodiments, the subject has a body weight of about 16 kg and arimoclomol citrate is administered at a dose of about 75 mg t.i.d. (i.e., about 225 mg/day). In some embodiments, the subject has a body weight of about 17 kg and arimoclomol citrate is administered at a dose of about 75 mg t.i.d. (i.e., about 225 mg/day). In some embodiments, the subject has a body weight of about 18 kg and arimoclomol citrate is administered at a dose of about 75 mg t.i.d. (i.e., about 225 mg/day). In some embodiments, the subject has a body weight of about 19 kg and arimoclomol citrate is administered at a dose of about 75 mg t.i.d. (i.e., about 225 mg/day). In some embodiments, the subject has a body weight of about 20 kg and arimoclomol citrate is administered at a dose of about 75 mg t.i.d. (i.e., about 225 mg/day). In some embodiments, the subject has a body weight of about 21 kg and arimoclomol citrate is administered at a dose of about 75 mg t.i.d. (i.e., about 225 mg/day). In some embodiments, the subject has a body weight of about 22 kg and arimoclomol citrate is administered at a dose of about 75 mg t.i.d. (i.e., about 225 mg/day).

In some embodiments, the subject has a body weight of about 22 kg to about 38 kg and arimoclomol citrate is administered at a dose of about 100 mg t.i.d. (i.e., about 300 mg/day).

In some embodiments, the subject has a body weight of about 22 kg and arimoclomol citrate is administered at a dose of about 100 mg t.i.d. (i.e., about 300 mg/day). In some embodiments, the subject has a body weight of about 23 kg and arimoclomol citrate is administered at a dose of about 100 mg t.i.d. (i.e., about 300 mg/day). In some embodiments, the subject has a body weight of about 24 kg and arimoclomol citrate is administered at a dose of about 100 mg t.i.d. (i.e., about 300 mg/day). In some embodiments, the subject has a body weight of about 25 kg and arimoclomol citrate is administered at a dose of about 100 mg t.i.d. (i.e., about 300 mg/day). In some embodiments, the subject has a body weight of about 26 kg and arimoclomol citrate is administered at a dose of about 100 mg t.i.d. (i.e., about 300 mg/day). In some embodiments, the subject has a body weight of about 27 kg and arimoclomol citrate is administered at a dose of about 100 mg t.i.d. (i.e., about 300 mg/day). In some embodiments, the subject has a body weight of about 28 kg and arimoclomol citrate is administered at a dose of about 100 mg t.i.d. (i.e., about 300 mg/day). In some embodiments, the subject has a body weight of about 29 kg and arimoclomol citrate is administered at a dose of about 100 mg t.i.d. (i.e., about 300 mg/day). In some embodiments, the subject has a body weight of about 30 kg and arimoclomol citrate is administered at a dose of about 100 mg t.i.d. (i.e., about 300 mg/day). In some embodiments, the subject has a body weight of about 31 kg and arimoclomol citrate is administered at a dose of about 100 mg t.i.d. (i.e., about 300 mg/day). In some embodiments, the subject has a body weight of about 32 kg and arimoclomol citrate is administered at a dose of about 100 mg t.i.d. (i.e., about 300 mg/day). In some embodiments, the subject has a body weight of about 33 kg and arimoclomol citrate is administered at a dose of about 100 mg t.i.d. (i.e., about 300 mg/day). In some embodiments, the subject has a body weight of about 34 kg and arimoclomol citrate is administered at a dose of about 100 mg t.i.d. (i.e., about 300 mg/day). In some embodiments, the subject has a body weight of about 35 kg and arimoclomol citrate is administered at a dose of about 100 mg t.i.d. (i.e., about 300 mg/day). In some embodiments, the subject has a body weight of about 36 kg and arimoclomol citrate is administered at a dose of about 100 mg t.i.d. (i.e., about 300 mg/day). In some embodiments, the subject has a body weight of about 37 kg and arimoclomol citrate is administered at a dose of about 100 mg t.i.d. (i.e., about 300 mg/day). In some embodiments, the subject has a body weight of about 38 kg and arimoclomol citrate is administered at a dose of about 100 mg t.i.d. (i.e., about 300 mg/day).

In some embodiments, the subject has a body weight of about 38 kg to about 55 kg and arimoclomol citrate is administered at a dose of about 150 mg t.i.d. (i.e., about 450 mg/day).

In some embodiments, the subject has a body weight of about 38 kg and arimoclomol citrate is administered at a dose of about 150 mg t.i.d. (i.e., about 450 mg/day). In some embodiments, the subject has a body weight of about 39 kg and arimoclomol citrate is administered at a dose of about 150 mg t.i.d. (i.e., about 450 mg/day). In some embodiments, the subject has a body weight of about 40 kg and arimoclomol citrate is administered at a dose of about 150 mg t.i.d. (i.e., about 450 mg/day). In some embodiments, the subject has a body weight of about 41 kg and arimoclomol citrate is administered at a dose of about 150 mg t.i.d. (i.e., about 450 mg/day). In some embodiments, the subject has a body weight of about 42 kg and arimoclomol citrate is administered at a dose of about 150 mg t.i.d. (i.e., about 450 mg/day). In some embodiments, the subject has a body weight of about 43 kg and arimoclomol citrate is administered at a dose of about 150 mg t.i.d. (i.e., about 450 mg/day). In some embodiments, the subject has a body weight of about 44 kg and arimoclomol citrate is administered at a dose of about 150 mg t.i.d. (i.e., about 450 mg/day). In some embodiments, the subject has a body weight of about 45 kg and arimoclomol citrate is administered at a dose of about 150 mg t.i.d. (i.e., about 450 mg/day). In some embodiments, the subject has a body weight of about 46 kg and arimoclomol citrate is administered at a dose of about 150 mg t.i.d. (i.e., about 450 mg/day). In some embodiments, the subject has a body weight of about 47 kg and arimoclomol citrate is administered at a dose of about 150 mg t.i.d. (i.e., about 450 mg/day). In some embodiments, the subject has a body weight of about 48 kg and arimoclomol citrate is administered at a dose of about 150 mg t.i.d. (i.e., about 450 mg/day). In some embodiments, the subject has a body weight of about 49 kg and arimoclomol citrate is administered at a dose of about 150 mg t.i.d. (i.e., about 450 mg/day). In some embodiments, the subject has a body weight of about 50 kg and arimoclomol citrate is administered at a dose of about 150 mg t.i.d. (i.e., about 450 mg/day). In some embodiments, the subject has a body weight of about 51 kg and arimoclomol citrate is administered at a dose of about 150 mg t.i.d. (i.e., about 450 mg/day). In some embodiments, the subject has a body weight of about 52 kg and arimoclomol citrate is administered at a dose of about 150 mg t.i.d. (i.e., about 450 mg/day). In some embodiments, the subject has a body weight of about 53 kg and arimoclomol citrate is administered at a dose of about 150 mg t.i.d. (i.e., about 450 mg/day). In some embodiments, the subject has a body weight of about 54 kg and arimoclomol citrate is administered at a dose of about 150 mg t.i.d. (i.e., about 450 mg/day). In some embodiments, the subject has a body weight of about 55 kg and arimoclomol citrate is administered at a dose of about 150 mg t.i.d. (i.e., about 450 mg/day).

In some embodiments, the subject has a body weight of great than about 55 kg and arimoclomol citrate is administered at a dose of about 200 mg t.i.d. (about 600 mg/day).

In some embodiments, miglustat is administered from about 300 mg/day to about 1000 mg/day. In some embodiments, miglustat is administered from about 350 mg/day to about 1000 mg/day. In some embodiments, miglustat is administered from about 400 mg/day to about 1000 mg/day. In some embodiments, miglustat is administered from about 450 mg/day to about 1000 mg/day. In some embodiments, miglustat is administered from about 500 mg/day to about 1000 mg/day. In some embodiments, miglustat is administered from about 550 mg/day to about 1000 mg/day. In some embodiments, miglustat is administered from about 600 mg/day to about 1000 mg/day. In some embodiments, miglustat is administered from about 650 mg/day to about 1000 mg/day. In some embodiments, miglustat is administered from about 700 mg/day to about 1000 mg/day. In some embodiments, miglustat is administered from about 750 mg/day to about 1000 mg/day. In some embodiments, miglustat is administered from about 800 mg/day to about 1000 mg/day. In some embodiments, miglustat is administered from about 850 mg/day to about 1000 mg/day. In some embodiments, miglustat is administered from about 900 mg/day to about 1000 mg/day. In some embodiments, miglustat is administered from about 950 mg/day to about 1000 mg/day.

In some embodiments, miglustat is administered from about 300 mg/day to about 950 mg/day. In some embodiments, miglustat is administered from about 300 mg/day to about 900 mg/day. In some embodiments, miglustat is administered from about 300 mg/day to about 850 mg/day. In some embodiments, miglustat is administered from about 300 mg/day to about 800 mg/day. In some embodiments, miglustat is administered from about 300 mg/day to about 750 mg/day. In some embodiments, miglustat is administered from about 300 mg/day to about 700 mg/day. In some embodiments, miglustat is administered from about 300 mg/day to about 650 mg/day. In some embodiments, miglustat is administered from about 300 mg/day to about 600 mg/day. In some embodiments, miglustat is administered from about 300 mg/day to about 550 mg/day. In some embodiments, miglustat is administered from about 300 mg/day to about 500 mg/day. In some embodiments, miglustat is administered from about 300 mg/day to about 450 mg/day. In some embodiments, miglustat is administered from about 300 mg/day to about 400 mg/day. In some embodiments, miglustat is administered from about 300 mg/day to about 350 mg/day.

In some embodiments, miglustat is administered at about 300 mg/day, about 325 mg/day, about 350 mg/day, about 375 mg/day, about 400 mg/day, about 425 mg/day, about 450 mg/day, about 475 mg/day, about 500 mg/day, about 525 mg/day, about 550 mg/day, about 575 mg/day, about 600 mg/day, about 625 mg/day, about 650 mg/day, about 675 mg/day, about 700 mg/day, about 725 mg/day, about 750 mg/day, about 775 mg/day, about 800 mg/day, about 825 mg/day, about 850 mg/day, about 875 mg/day, about 900 mg/day, about 925 mg/day, about 950 mg/day, about 975 mg/day, or about 1000 mg/day.

In some embodiments, miglustat is administered at about 300 mg/day. In some embodiments, miglustat is administered at about 325 mg/day. In some embodiments, miglustat is administered at about 350 mg/day. In some embodiments, miglustat is administered at about 375 mg/day. In some embodiments, miglustat is administered at about 400 mg/day. In some embodiments, miglustat is administered at about 425 mg/day. In some embodiments, miglustat is administered at about 450 mg/day. In some embodiments, miglustat is administered at about 475 mg/day. In some embodiments, miglustat is administered at about 500 mg/day. In some embodiments, miglustat is administered at about 525 mg/day. In some embodiments, miglustat is administered at about 550 mg/day. In some embodiments, miglustat is administered at about 575 mg/day. In some embodiments, miglustat is administered at about 600 mg/day. In some embodiments, miglustat is administered at about 625 mg/day. In some embodiments, miglustat is administered at about 650 mg/day. In some embodiments, miglustat is administered at about 675 mg/day. In some embodiments, miglustat is administered at about 700 mg/day. In some embodiments, miglustat is administered at about 725 mg/day. In some embodiments, miglustat is administered at about 750 mg/day. In some embodiments, miglustat is administered at about 775 mg/day. In some embodiments, miglustat is administered at about 800 mg/day. In some embodiments, miglustat is administered at about 825 mg/day. In some embodiments, miglustat is administered at about 850 mg/day. In some embodiments, miglustat is administered at about 875 mg/day. In some embodiments, miglustat is administered at about 900 mg/day. In some embodiments, miglustat is administered at about 925 mg/day. In some embodiments, miglustat is administered at about 950 mg/day. In some embodiments, miglustat is administered at about 975 mg/day. In some embodiments, miglustat is administered at about 1000 mg/day.

In some embodiments, miglustat is administered at about 100 mg to about 300 mg. In some embodiments, miglustat is administered at about 125 mg to about 300 mg. In some embodiments, miglustat is administered at about 150 mg to about 300 mg. In some embodiments, miglustat is administered at about 175 mg to about 300 mg. In some embodiments, miglustat is administered at about 200 mg to about 300 mg. In some embodiments, miglustat is administered at about 225 mg to about 300 mg. In some embodiments, miglustat is administered at about 250 mg to about 300 mg. In some embodiments, miglustat is administered at about 275 mg to about 300 mg.

In some embodiments, miglustat is administered at about 100 mg to about 275 mg. In some embodiments, miglustat is administered at about 100 mg to about 250 mg. In some embodiments, miglustat is administered at about 100 mg to about 225 mg. In some embodiments, miglustat is administered at about 100 mg to about 200 mg. In some embodiments, miglustat is administered at about 100 mg to about 175 mg. In some embodiments, miglustat is administered at about 100 mg to about 150 mg. In some embodiments, miglustat is administered at about 100 mg to about 125 mg.

In some embodiments, miglustat is administered at about 100 mg. In some embodiments, miglustat is administered at about 125 mg. In some embodiments, miglustat is administered at about 150 mg. In some embodiments, miglustat is administered at about 175 mg. In some embodiments, miglustat is administered at about 200 mg. In some embodiments, miglustat is administered at about 225 mg. In some embodiments, miglustat is administered at about 250 mg. In some embodiments, miglustat is administered at about 275 mg. In some embodiments, miglustat is administered at about 300 mg.

In some embodiments, miglustat dosage is adjusted for subjects under the age of 12 years on the basis of body surface area.

In some embodiments, arimoclomol is co-administered with miglustat.

In some embodiments, arimoclomol and miglustat are administered in temporal proximity.

In some embodiments, miglustat is administered prior to arimoclomol.

In some embodiments, arimoclomol is administered prior to miglustat.

In some embodiments, arimoclomol and miglustat are administered simultaneously or sequentially.

In some embodiments, miglustat is administered for at least six-months prior to the first dose of arimoclomol.

In some embodiments, miglustat is administered for at least one year prior to the first dose of arimoclomol. In some embodiments, miglustat is administered for at least two years prior to the first dose of arimoclomol.

In some embodiments, arimoclomol citrate is co-administered with miglustat.

In some embodiments, arimoclomol citrate and miglustat are administered in temporal proximity.

In some embodiments, miglustat is administered prior to arimoclomol citrate.

In some embodiments, arimoclomol citrate is administered prior to miglustat.

In some embodiments, arimoclomol citrate and miglustat are administered simultaneously or sequentially.

In some embodiments, miglustat is administered for at least six-months prior to the first dose of arimoclomol citrate.

In some embodiments, miglustat is administered for at least one year prior to the first dose of arimoclomol citrate. In some embodiments, miglustat is administered for at least two years prior to the first dose of arimoclomol citrate.

In some embodiments, the administration of arimoclomol reduces accumulation of unesterified cholesterol. In some embodiments, the reduction of unesterified cholesterol occurs in peripheral blood mononuclear cells (PBMCs). In some aspects, the administration of arimoclomol reduces unesterified cholesterol such that any increase in unesterified cholesterol exhibited by the subject is no more than about 10,000 ng/mg protein, or no more than about 15,000 ng/mg protein, or no more than about 20,000 ng/mg protein, or more than about 25,000 ng/mg protein, or no more than about 30,000 ng/mg protein, or nor more than about 35,000 ng/mg protein, or no more than about 40,000 ng/mg protein.

In some embodiments, the administration of arimoclomol citrate reduces accumulation of unesterified cholesterol. In some aspects, the administration of arimoclomol citrate reduces unesterified cholesterol such that any increase in unesterified cholesterol exhibited by the subject is no more than about 10,000 ng/mg protein, or no more than about 15,000 ng/mg protein, or no more than about 20,000 ng/mg protein, or more than about 25,000 ng/mg protein, or no more than about 30,000 ng/mg protein, or nor more than about 35,000 ng/mg protein, or no more than about 40,000 ng/mg protein.

In some embodiments, the administration of arimoclomol reduces accumulation of serum cholestane-triol levels. In some embodiments, the administration of arimoclomol reduces the accumulation of serum cholestane-triol levels such that there is a decrease of at least about 2.5 ng/ml, or a decrease of at least about 3.0 ng/ml, or a decrease of at least about 3.5 ng/ml, or a decrease of at least about 4.0 ng/ml, or a decrease of at least about 4.5 ng/ml, or a decrease of at least about 5.0 ng/ml, or a decrease of at least about 5.5 ng/ml, or a decrease of at least about 6.0 ng/ml, or a decrease of at least about 6.5 ng/ml, or a decrease of at least about 7.0 ng/ml, or a decrease of at least about 7.5 ng/ml, or a decrease of at least about 7.5 ng/ml, or a decrease of at least about 8.0 ng/ml, or a decrease of at least about 8.5 ng/ml, or a decrease of at least about 9.0 ng/ml, or a decrease of at least about 9.5 ng/ml, or a decrease of at least about 10.0 ng/ml.

In some embodiments, the administration of arimoclomol citrate reduces accumulation of serum cholestane-triol levels. In some embodiments, the administration of arimoclomol citrate reduces the accumulation of serum cholestane-triol levels such that there is a decrease of at least about 2.5 ng/ml, or a decrease of at least about 3.0 ng/ml, or a decrease of at least about 3.5 ng/ml, or a decrease of at least about 4.0 ng/ml, or a decrease of at least about 4.5 ng/ml, or a decrease of at least about 5.0 ng/ml, or a decrease of at least about 5.5 ng/ml, or a decrease of at least about 6.0 ng/ml, or a decrease of at least about 6.5 ng/ml, or a decrease of at least about 7.0 ng/ml, or a decrease of at least about 7.5 ng/ml, or a decrease of at least about 7.5 ng/ml, or a decrease of at least about 8.0 ng/ml, or a decrease of at least about 8.5 ng/ml, or a decrease of at least about 9.0 ng/ml, or a decrease of at least about 9.5 ng/ml, or a decrease of at least about 10.0 ng/ml.

In some embodiments, the administration of arimoclomol modifies the course of NPC such that the subject who is administered arimoclomol exhibits an increase in NPCCSS score that is no more than about 0.1, or about 0.2, or about 0.3, or about 0.4, or about 0.5, or about 0.6, or about 0.7, or about 0.8, or about 0.9, or about 1.0, or about 1.1, or about 1.2, or about 1.3, or about 1.4, or about 1.5, or about 1.6, or about 1.7, or about 1.8, or about 1.9, or about 2.0 over the course of treatment. In some embodiments, the course of treatment can be a period of at least about 1 month, or about 2 months, or about 3 months, or about 4 months, or about 5 months, or about 6 months, or about 7 months, or about 8 months, or about 9 months, or about 10 months, or about 11 months, or about 12 months, or about 13 months, or about 14 months, or about 15 months, or about 16 months, or about 17 months, or about 18 months, or about 19 months, or about 20 months, or about 21 months, or about 22 months, or about 23 months, or about 24 months. In some embodiments, the course of treatment can be a period greater than 24 months. The NPCCSS score can be a 5-domain NPCCSS score or a 17-domain NPCCSS score, as described herein.

In some embodiments, the administration of arimoclomol citrate modifies the course of NPC such that the subject who is administered arimoclomol citrate exhibits an increase in NPCCSS score that is no more than about 0.1, or about 0.2, or about 0.3, or about 0.4, or about 0.5, or about 0.6, or about 0.7, or about 0.8, or about 0.9, or about 1.0, or about 1.1, or about 1.2, or about 1.3, or about 1.4, or about 1.5, or about 1.6, or about 1.7, or about 1.8, or about 1.9, or about 2.0 over the course of treatment.

Definitions

Unless explicitly indicated otherwise, the terms “arimoclomol”, “N-[(2R)-2-hydroxy-3-piperidin-1-ylpropoxy]-1-oxidopyridin-1-ium-3-carboximidoyl chloride”, and “BRX-345” refer to the compound which has the following structure:

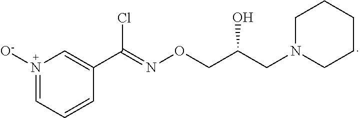

Arimoclomol, and its preparation are disclosed in PCT Application Publication Nos. WO 97/16439, WO 00/050403 and WO 01/79174. These publications are incorporated by reference herein in their entireties.

Unless explicitly indicated otherwise, any reference to “arimoclomol”, and “N-[(2R)-2-hydroxy-3-piperidin-1-ylpropoxy]-1-oxidopyridin-1-ium-3-carboximidoyl chloride” refers also to pharmaceutically acceptable salts of, and pharmaceutical compositions comprising, the compound which has the following structure:

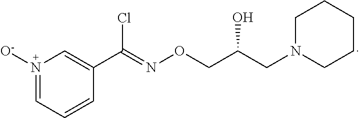

Unless explicitly indicated otherwise the terms “arimoclomol citrate”, “N-[(2R)-2-hydroxy-3-piperidin-1-ylpropoxy]-1-oxidopyridin-1-ium-3-carboximidoyl chloride citrate”, and “BRX-345” refer to the citrate salt of arimoclomol.

Unless explicitly indicated otherwise, the terms “miglustat”, “Zavesca®”, “Brazaves”, “OGT 918”, “N-Butylmoranoline”, “1,5-(butylimino)-1,5-dideoxy-D-glucitol”, “N-butyl-deoxynojirimycin”, and “(2R,3R,4R,5 S)-1-butyl-2-(hydroxymethyl)piperidine-3 ,4,5-triol” refer to the compound which has the following structure:

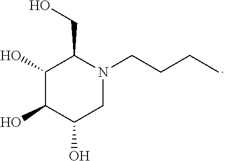

Unless explicitly indicated otherwise, any reference to “miglustat”, “Zavesca®”, “Brazaves”, “OGT 918”, “N-Butylmoranoline”, “1,5-(butylimino)-1,5-dideoxy-D-glucitol”, “N-butyl-deoxynojirimycin”, and “(2R,3R,4R,5 S)-1-butyl-2-(hydroxymethyl)piperidine-3 ,4,5-triol” refers also to pharmaceutically acceptable salts of, and pharmaceutical compositions comprising, the compound which has the following structure:

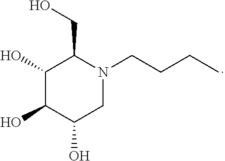

Niemann-Pick disease type C (NPC) is a rare progressive genetic disorder characterized by an inability of the body to transport cholesterol and other fatty substances (lipids) inside of cells. This leads to the abnormal accumulation of these substances within various tissues of the body, including brain tissue.

The term “early treatment initiation” refers to starting the administration of arimoclomol as early as possible in the course of NPC in a subject, for example, as soon as the subject has been diagnosed with NPC or during a time period in which the subject exhibits a 5-domain NPCCSS, 17-domain NPCCSS, and/or ASIS score that is below a certain predetermined cutoff value.

The term “NPC1” refers to the gene encoding the Niemann-Pick type C protein 1, also referred to in the art as the NPC intracellular cholesterol transporter 1. The term “NPC1” refers to the protein product of the NPC1 gene.

The term “NPC2” refers to the gene encoding the Niemann-Pick type C protein 2, also referred to in the art as the NPC intracellular cholesterol transporter 1. The term “NPC2” refers to the protein product of the NPC2 gene.

The term “genotype” refers to the mutational status of both of the alleles of a particular gene locus in a subject. As would be appreciated by the skilled artisan, genotypes are described herein by a mutational description (e.g. missense, functional null, specific mutation description) of the first allele, followed by a “/”, followed by a mutational description (e.g. missense, functional null, specific mutation description) of the second allele.

The term “NPC1 genotype” refers to the genotype of a subject at the NPC1 gene locus.

The term “NPC2 genotype” refers to the genotype of a subject at the NPC2 gene locus.

The term “functional null mutation” refers to a mutation in a gene that results in a truncated and defective protein being produced from the allele that has the functional null mutation. As used herein a functional null mutation is either a frameshift mutation, an aberrant splicing mutation, or a premature stop codon mutation.

The term “frameshift mutation” refers to either a deletion or insertion of any number of nucleotides an allele of a gene in a genome of a subject, wherein the number of nucleotides is not divisible by three, which causes a change in the reading frame of the transcript produced from the allele with the mutation as compared to the transcript produced from a wildtype allele.

The term “aberrant splicing mutation” refers to mutation that changes the splicing activity of a transcript containing the mutation as compared to a transcript with a wildtype sequence.

The term “premature stop mutation” refers to a mutation in a single nucleotide that leads to the creation of a premature stop codon, resulting in the production of a truncated protein product as compared to the wildtype sequence.

The term “missense mutation” refers to one or more nucleotide substitutions resulting in a gene that results in a single amino-acid change in the protein produced by the gene with the mutation as compared to the protein produced by the wildtype gene.

Mutations on both genome and protein level in this disclosure are labelled according to the 2016 standards of the Human Genome Variation Society (HGVS) and Human genome Organization (HUGO) nomenclature, as would be appreciated by the skilled artisan. DNA mutations are numbered relative to NPC1 cDNA. A single letter amino-acid code is used throughout, frameshift mutations are labelled is' after the first affected amino acid, splice mutations are indicated with ‘sp’ after the last presumed correctly translated amino acid and ‘*’ denotes a stop codon.

Mutation nomenclature used herein with respect to nucleotide positions refers to positions within the sequence described in National Library of Medicine entry NM 000271.5:

(SEQ ID NO: 1)
CTTCCTGACCGGCGCGCGCAGCCTGCTGCCGCGGTCAGCGCCTGCTCCTGCTCCTCCGCTCCTCC
TGCGCGGGGTGCTGAAACAGCCCGGGGAAGTAGAGCCGCCTCCGGGGAGCCCAACCAGCCGAACG
CCGCCGGCGTCAGCAGCCTTGCGCGGCCACAGCATGACCGCTCGCGGCCTGGCCCTTGGCCTCCT
CCTGCTGCTACTGTGTCCAGCGCAGGTGTTTTCACAGTCCTGTGTTTGGTATGGAGAGTGTGGAA
TTGCATATGGGGACAAGAGGTACAATTGCGAATATTCTGGCCCACCAAAACCATTGCCAAAGGAT
GGATATGACTTAGTGCAGGAACTCTGTCCAGGATTCTTCTTTGGCAATGTCAGTCTCTGTTGTGA
TGTTCGGCAGCTTCAGACACTAAAAGACAACCTGCAGCTGCCTCTACAGTTTCTGTCCAGATGTC
CATCCTGTTTTTATAACCTACTGAACCTGTTTTGTGAGCTGACATGTAGCCCTCGACAGAGTCAG
TTTTTGAATGTTACAGCTACTGAAGATTATGTTGATCCTGTTACAAACCAGACGAAAACAAATGT
GAAAGAGTTACAATACTACGTCGGACAGAGTTTTGCCAATGCAATGTACAATGCCTGCCGGGATG
TGGAGGCCCCCTCAAGTAATGACAAGGCCCTGGGACTCCTGTGTGGGAAGGACGCTGACGCCTGT
AATGCCACCAACTGGATTGAATACATGTTCAATAAGGACAATGGACAGGCACCTTTTACCATCAC
TCCTGTGTTTTCAGATTTTCCAGTCCATGGGATGGAGCCCATGAACAATGCCACCAAAGGCTGTG
ACGAGTCTGTGGATGAGGTCACAGCACCATGTAGCTGCCAAGACTGCTCTATTGTCTGTGGCCCC
AAGCCCCAGCCCCCACCTCCTCCTGCTCCCTGGACGATCCTTGGCTTGGACGCCATGTATGTCAT
CATGTGGATCACCTACATGGCGTTTTTGCTTGTGTTTTTTGGAGCATTTTTTGCAGTGTGGTGCT
ACAGAAAACGGTATTTTGTCTCCGAGTACACTCCCATCGATAGCAATATAGCTTTTTCTGTTAAT
GCAAGTGACAAAGGAGAGGCGTCCTGCTGTGACCCTGTCAGCGCAGCATTTGAGGGCTGCTTGAG
GCGGCTGTTCACACGCTGGGGGTCTTTCTGCGTCCGAAACCCTGGCTGTGTCATTTTCTTCTCGC
TGGTCTTCATTACTGCGTGTTCGTCAGGCCTGGTGTTTGTCCGGGTCACAACCAATCCAGTTGAC
CTCTGGTCAGCCCCCAGCAGCCAGGCTCGCCTGGAAAAAGAGTACTTTGACCAGCACTTTGGGCC
TTTCTTCCGGACGGAGCAGCTCATCATCCGGGCCCCTCTCACTGACAAACACATTTACCAGCCAT
ACCCTTCGGGAGCTGATGTACCCTTTGGACCTCCGCTTGACATACAGATACTGCACCAGGTTCTT
GACTTACAAATAGCCATCGAAAACATTACTGCCTCTTATGACAATGAGACTGTGACACTTCAAGA
CATCTGCTTGGCCCCTCTTTCACCGTATAACACGAACTGCACCATTTTGAGTGTGTTAAATTACT
TCCAGAACAGCCATTCCGTGCTGGACCACAAGAAAGGGGACGACTTCTTTGTGTATGCCGATTAC
CACACGCACTTTCTGTACTGCGTACGGGCTCCTGCCTCTCTGAATGATACAAGTTTGCTCCATGA
CCCTTGTCTGGGTACGTTTGGTGGACCAGTGTTCCCGTGGCTTGTGTTGGGAGGCTATGATGATC
AAAACTACAATAACGCCACTGCCCTTGTGATTACCTTCCCTGTCAATAATTACTATAATGATACA
GAGAAGCTCCAGAGGGCCCAGGCCTGGGAAAAAGAGTTTATTAATTTTGTGAAAAACTACAAGAA
TCCCAATCTGACCATTTCCTTCACTGCTGAACGAAGTATTGAAGATGAACTAAATCGTGAAAGTG
ACAGTGATGTCTTCACCGTTGTAATTAGCTATGCCATCATGTTTCTATATATTTCCCTAGCCTTG
GGGCACATGAAAAGCTGTCGCAGGCTTCTGGTGGATTCGAAGGTCTCACTAGGCATCGCGGGCAT
CTTGATCGTGCTGAGCTCGGTGGCTTGCTCCTTGGGTGTCTTCAGCTACATTGGGTTGCCCTTGA
CCCTCATTGTGATTGAAGTCATCCCGTTCCTGGTGCTGGCTGTTGGAGTGGACAACATCTTCATT
CTGGTGCAGGCCTACCAGAGAGATGAACGTCTTCAAGGGGAAACCCTGGATCAGCAGCTGGGCAG
GGTCCTAGGAGAAGTGGCTCCCAGTATGTTCCTGTCATCCTTTTCTGAGACTGTAGCATTTTTCT
TAGGAGCATTGTCCGTGATGCCAGCCGTGCACACCTTCTCTCTCTTTGCGGGATTGGCAGTCTTC
ATTGACTTTCTTCTGCAGATTACCTGTTTCGTGAGTCTCTTGGGGTTAGACATTAAACGTCAAGA
GAAAAATCGGCTAGACATCTTTTGCTGTGTCAGAGGTGCTGAAGATGGAACAAGCGTCCAGGCCT
CAGAGAGCTGTTTGTTTCGCTTCTTCAAAAACTCCTATTCTCCACTTCTGCTAAAGGACTGGATG
AGACCAATTGTGATAGCAATATTTGTGGGTGTTCTGTCATTCAGCATCGCAGTCCTGAACAAAGT
AGATATTGGATTGGATCAGTCTCTTTCGATGCCAGATGACTCCTACATGGTGGATTATTTCAAAT
CCATCAGTCAGTACCTGCATGCGGGTCCGCCTGTGTACTTTGTCCTGGAGGAAGGGCACGACTAC
ACTTCTTCCAAGGGGCAGAACATGGTGTGCGGCGGCATGGGCTGCAACAATGATTCCCTGGTGCA
GCAGATATTTAACGCGGCGCAGCTGGACAACTATACCCGAATAGGCTTCGCCCCCTCGTCCTGGA
TCGACGATTATTTCGACTGGGTGAAGCCACAGTCGTCTTGCTGTCGAGTGGACAATATCACTGAC
CAGTTCTGCAATGCTTCAGTGGTTGACCCTGCCTGCGTTCGCTGCAGGCCTCTGACTCCGGAAGG
CAAACAGAGGCCTCAGGGGGGAGACTTCATGAGATTCCTGCCCATGTTCCTTTCGGATAACCCTA
ACCCCAAGTGTGGCAAAGGGGGACATGCTGCCTATAGTTCTGCAGTTAACATCCTCCTTGGCCAT
GGCACCAGGGTCGGAGCCACGTACTTCATGACCTACCACACCGTGCTGCAGACCTCTGCTGACTT
TATTGACGCTCTGAAGAAAGCCCGACTTATAGCCAGTAATGTCACCGAAACCATGGGCATTAACG
GCAGTGCCTACCGAGTATTTCCTTACAGTGTGTTTTATGTCTTCTACGAACAGTACCTGACCATC
ATTGACGACACTATCTTCAACCTCGGTGTGTCCCTGGGCGCGATATTTCTGGTGACCATGGTCCT
CCTGGGCTGTGAGCTCTGGTCTGCAGTCATCATGTGTGCCACCATCGCCATGGTCTTGGTCAACA
TGTTTGGAGTTATGTGGCTCTGGGGCATCAGTCTGAACGCTGTATCCTTGGTCAACCTGGTGATG
AGCTGTGGCATCTCCGTGGAGTTCTGCAGCCACATAACCAGAGCGTTCACGGTGAGCATGAAAGG
CAGCCGCGTGGAGCGCGCGGAAGAGGCACTTGCCCACATGGGCAGCTCCGTGTTCAGTGGAATCA
CACTTACAAAATTTGGAGGGATTGTGGTGTTGGCTTTTGCCAAATCTCAAATTTTCCAGATATTC
TACTTCAGGATGTATTTGGCCATGGTCTTACTGGGAGCCACTCACGGATTAATATTTCTCCCTGT
CTTACTCAGTTACATAGGGCCATCAGTAAATAAAGCCAAAAGTTGTGCCACTGAAGAGCGATACA
AAGGAACAGAGCGCGAACGGCTTCTAAATTTCTAGCCCTCTCGCAGGGCATCCTGACTGAACTGT
GTCTAAGGGTCGGTCGGTTTACCACTGGACGGGTGCTGCATCGGCAAGGCCAAGTTGAACACCGG
ATGGTGCCAACCATCGGTTGTTTGGCAGCAGCTTTGAACGTAGCGCCTGTGAACTCAGGAATGCA
CAGTTGACTTGGGAAGCAGTATTACTAGATCTGGAGGCAACCACAGGACACTAAACTTCTCCCAG
CCTCTTCAGGAAAGAAACCTCATTCTTTGGCAAGCAGGAGGTGACACTAGATGGCTGTGAATGTG
ATCCGCTCACTGACACTCTGTAAAGGCCAATCAATGCACTGTCTGTCTCTCCTTTTAGGAGTAAG
CCATCCCACAAGTTCTATACCATATTTTTAGTGACAGTTGAGGTTGTAGATACACTTTATAACAT
TTTATAGTTTAAAGAGCTTTATTAATGCAATAAATTAACTTTGTACACATTTTTATATAAAAAAA
CAGCAAGTGATTTCAGAATGTTGTAGGCCTCATTAGAGCTTGGTCTCCAAAAATCTGTTTGAAAA
AAGCAACATGTTCTTCACAGTGTTCCCCTAGAAAGGAAGAGATTTAATTGCCAGTTAGATGTGGC
ATGAAATGAGGGACAAAGAAAGCATCTCGTAGGTGTGTCTACTGGGTTTTAACTTATTTTTCTTT
AATAAAATACATTGTTTTCCTAAGTTTTGGGGTTACCCTATCTGCTTTGAGAGACAAATACAAAA
GCTAAATGGAAGAGA

Mutation nomenclature used herein with respect to amino acid positions refers to positions within the sequence described in National Library of Medicine entry NP_000262.2:

(SEQ ID NO: 2)
MTARGLALGLLLLLLCPAQVFSQSCVWYGECGIAYGDKRYNCEYSGPPKP
LPKDGYDLVQELCPGFFFGNVSLCCDVRQLQTLKDNLQLPLQFLSRCPSC
FYNLLNLFCELTCSPRQSQFLNVTATEDYVDPVTNQTKTNVKELQYYVGQ
SFANAMYNACRDVEAPSSNDKALGLLCGKDADACNATNWIEYMFNKDNGQ
APFTITPVFSDFPVHGMEPMNNATKGCDESVDEVTAPCSCQDCSIVCGPK
PQPPPPPAPWTILGLDAMYVIMWITYMAFLLVFFGAFFAVWCYRKRYFVS
EYTPIDSNIAFSVNASDKGEASCCDPVSAAFEGCLRRLFTRWGSFCVRNP
GCVIFFSLVFITACSSGLVFVRVTTNPVDLWSAPSSQARLEKEYFDQHFG
PFFRTEQLIIRAPLTDKHIYQPYPSGADVPFGPPLDIQILHQVLDLQIAI
ENITASYDNETVTLQDICLAPLSPYNTNCTILSVLNYFQNSHSVLDHKKG
DDFFVYADYHTHFLYCVRAPASLNDTSLLHDPCLGTFGGPVFPWLVLGGY
DDQNYNNATALVITFPVNNYYNDTEKLQRAQAWEKEFINFVKNYKNPNLT
ISFTAERSIEDELNRESDSDVFTVVISYAIMFLYISLALGHMKSCRRLLV
DSKVSLGIAGILIVLSSVACSLGVFSYIGLPLTLIVIEVIPFLVLAVGVD
NIFILVQAYQRDERLQGETLDQQLGRVLGEVAPSMFLSSFSETVAFFLGA
LSVMPAVHTFSLFAGLAVFIDFLLQITCFVSLLGLDIKRQEKNRLDIFCC
VRGAEDGTSVQASESCLFRFFKNSYSPLLLKDWMRPIVIAIFVGVLSFSI
AVLNKVDIGLDQSLSMPDDSYMVDYFKSISQYLHAGPPVYFVLEEGHDYT
SSKGQNMVCGGMGCNNDSLVQQIFNAAQLDNYTRIGFAPSSWIDDYFDWV
KPQSSCCRVDNITDQFCNASVVDPACVRCRPLTPEGKQRPQGGDFMRFLP
MFLSDNPNPKCGKGGHAAYSSAVNILLGHGTRVGATYFMTYHTVLQTSAD
FIDALKKARLIASNVTETMGINGSAYRVFPYSVFYVFYEQYLTIIDDTIF
NLGVSLGAIFLVTMVLLGCELWSAVIMCATIAMVLVNMFGVMWLWGISLN
AVSLVNLVMSCGISVEFCSHITRAFTVSMKGSRVERAEEALAHMGSSVFS
GITLTKFGGIVVLAFAKSQIFQIFYFRMYLAMVLLGATHGLIFLPVLLSY
IGPSVNKAKSCATEERYKGTERERLLNF

The term “ER type missense mutation” refers to a missense mutation that results in production of an NPC protein (e.g. NPC1 or NPC2) that is misfolded, retained at the endoplasmic reticulum (ER) and subsequently targeted for degradation.

The term “compound heterozygote” refers to an individual that has two different mutant alleles at a particular gene locus (e.g. at an NPC gene locus such as the NPC1 locus or the NPC2 locus). In a non-limiting example, a subject who has an NPC genotype of Missense/Missense would be considered a compound heterozygote if the subject had a first missense mutation on the first NPC allele and a second missense mutation on the second NPC allele, wherein the first missense mutation and the second missense mutation are different.

Unless explicitly indicated otherwise, the terms “approximately” and “about” are synonymous. In some embodiments, “approximately” and “about” refer to the recited amount, value, or duration ±5%, ±4.5%, ±4%, ±3.5%, ±3%, ±2.5%, ±2%, ±1.75%, ±1.5%, ±1.25%, ±1%, ±0.9%, ±0.8%, ±0.7%, ±0.6%, ±0.5% ±0.4%, ±0.3%, ±0.2%, ±0.1%, ±0.09%, ±0.08%, ±0.07%, ±0.06%, ±0.05%, ±0.04%, ±0.03%, ±0.02%, or ±0.01%. In some embodiments, “approximately” and “about” refer to the listed amount, value, or duration ±2.5%, ±2%, ±1.75%, ±1.5%, ±1.25%, ±1%, ±0.9%, ±0.8%, ±0.7%, ±0.6%, ±0.5%. In some embodiments, “approximately” and “about” refer to the listed amount, value, or duration ±1%. In some embodiments, “approximately” and “about” refer to the listed amount, value, or duration ±0.5%. In some embodiments, “approximately” and “about” refer to the listed amount, value, or duration ±0.1%.

The term “subject” includes any living organism that has NPC, or is at a risk of developing NPC. In some embodiments, the term “subject” refers to a mammal that has NPC, or is at a risk of developing NPC. In some embodiments, the term subject refers to a human being that has NPC, or is at a risk of developing NPC. The term “patient” is meant to be synonymous and may be used interchangeably with “subject,” unless explicitly indicated otherwise.

As used herein, the term “treating” or “treat” describes the management and care of a subject for the purpose of combating a disease, condition, or disorder and includes the administration of a compound of the present disclosure, or a pharmaceutically acceptable salt, polymorph or solvate thereof, to alleviate the symptoms or complications of a disease, condition or disorder, or to eliminate the disease, condition or disorder. The term “treat” can also include treatment of a cell in vitro or an animal model.

It is to be appreciated that references to “treating” or “treatment” include the alleviation of established symptoms of a condition. “Treating” or “treatment” of a state, disorder or condition therefore includes: (1) delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

As used herein, the term “preventing,” “prevent,” or “protecting against” describes reducing or eliminating the onset of the symptoms or complications of such disease, condition or disorder.

The term “therapeutically effective amount”, as used herein, refers to an amount of a pharmaceutical agent, e.g., arimoclomol, to treat, ameliorate, or prevent an identified disease or condition, e.g., NPC, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician.

For any compound, the therapeutically effective amount can be estimated in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD50 (the dose lethal to 50% of the population). The dose ratio between toxic and therapeutic effects is the therapeutic index, and it can be expressed as the ratio, LD₅₀/ED₅₀. The dosage may vary within this range depending upon the dosage form employed and sensitivity of the subject.

The terms “administer”, “administering”, “administration”, and the like, as used herein, refer to methods that may be used to enable delivery of compositions to the desired site of biological action. These methods include, but are not limited to, intraarticular (in the joints), intravenous, intramuscular, intratumoral, intradermal, intraperitoneal, subcutaneous, orally, topically, intrathecally, inhalationally, transdermally, rectally, and the like. Administration techniques that can be employed with the agents and methods described herein are found in e.g., Goodman and Gilman, The Pharmacological Basis of Therapeutics, current ed.; Pergamon; and Remington's, Pharmaceutical Sciences (current edition), Mack Publishing Co., Easton, Pa.

In addition, the arimoclomol can be co-administered with other therapeutic agents. As used herein, the terms “co-administration”, “administered in combination with”, “administered in temporal proximity”, and their grammatical equivalents, are meant to encompass administration of two or more therapeutic agents to a single subject, and are intended to include treatment regimens in which the agents are administered by the same or different route of administration or at the same or different times. In some embodiments arimoclomol will be co-administered with other agents. These terms encompass administration of two or more agents to the subject so that both agents and/or their metabolites are present in the subject at the same time. They include simultaneous administration in separate compositions, administration at different times in separate compositions, and/or administration in a composition in which both agents are present. Thus, in some embodiments, the compounds described herein and the other agent(s) are administered in a single composition. In some embodiments, the compounds described herein and the other agent(s) are admixed in the composition.

The particular mode of administration and the dosage regimen will be selected by the attending clinician, taking into account the particulars of the case (e.g., the subject, the disease, the disease state involved, the particular treatment). Treatment can involve daily or multi-daily or less than daily (such as weekly or monthly etc.) doses over a period of a few days to months, or even years. However, a person of ordinary skill in the art would immediately recognize appropriate and/or equivalent doses looking at dosages of approved compositions for treating NPC using arimoclomol for guidance.

The compounds or the corresponding pharmaceutical compositions taught herein can be administered to a patient in a variety of forms depending on the selected route of administration, as will be understood by those skilled in the art. The compounds of the present teachings may be administered, for example, by oral, parenteral, buccal, sublingual, nasal, rectal, patch, pump or transdermal administration and the pharmaceutical compositions formulated accordingly. Parenteral administration includes intravenous, intraperitoneal, subcutaneous, intramuscular, transepithelial, nasal, intrapulmonary, intrathecal, rectal and topical modes of administration. Parenteral administration can be by continuous infusion over a selected period of time.

The pharmaceutical composition of the application is formulated to be compatible with its intended route of administration. In some embodiments, the composition is formulated in accordance with routine procedures as a pharmaceutical composition adapted for intravenous, subcutaneous, intramuscular, oral, intranasal, or topical administration to human beings. In preferred embodiments, the pharmaceutical composition is formulated for intravenous administration.

In some embodiments, the oral therapeutic administration may be incorporated with an excipient and used in the form of ingestible tablets, buccal tablets, troches, capsules, elixirs, suspensions, syrups, wafers, and the like.

In some embodiments, the parenteral administration may be generally prepared in water suitably mixed with a surfactant such as hydroxypropylcellulose. Dispersions can also be prepared in glycerol, liquid polyethylene glycols, DMSO and mixtures thereof with or without alcohol, and in oils. Under ordinary conditions of storage and use, these preparations contain a preservative to prevent the growth of microorganisms.

In some embodiments, the injectable administration may be prepared as a sterile aqueous solution or dispersion thereof, and sterile powders of, arimoclomol for the extemporaneous preparation of sterile injectable solutions or dispersions are appropriate.

### EXEMPLARY EMBODIMENTS

Embodiment 1. A method of treating or preventing Niemann Pick disease, type C (NPC) in a subject in need thereof, the method comprising administering a therapeutically effective amount of arimoclomol to the subject, wherein the subject has an NPC genotype of either Missense/Missense or Missense/Functional Null.

Embodiment la. A method of treating Niemann Pick disease, type C (NPC) in a subject in need thereof, the method comprising administering a therapeutically effective amount of arimoclomol to the subject, wherein the subject has an NPC genotype of either Missense/Missense or Missense/Functional Null.

Embodiment 2. Use of Arimoclomol in the manufacture of a medicament for treating or preventing Niemann Pick disease, type C (NPC) in a subject in need thereof, wherein the subject has an NPC genotype of either Missense/Missense or Missense/Functional Null.

Embodiment 2a. Use of Arimoclomol in the manufacture of a medicament for treating Niemann Pick disease, type C (NPC) in a subject in need thereof, wherein the subject has an NPC genotype of either Missense/Missense or Missense/Functional Null.

Embodiment 3. Arimoclomol for use in the treatment or prevention of Niemann Pick disease, type C (NPC) in a subject in need thereof, wherein the subject has an NPC genotype of either Missense/Missense or Missense/Functional Null.

Embodiment 3a. Arimoclomol for use in the treatment of Niemann Pick disease, type C (NPC) in a subject in need thereof, wherein the subject has an NPC genotype of either Missense/Missense or Missense/Functional Null.

Embodiment 4. A method of treating or preventing Niemann Pick disease, type C (NPC) in a subject in need thereof, the method comprising administering a therapeutically effective amount of arimoclomol and a therapeutically effective amount of miglustat to the subject, wherein the subject has an NPC genotype of either Missense/Missense or Missense/Functional Null.

Embodiment 4a. A method of treating Niemann Pick disease, type C (NPC) in a subject in need thereof, the method comprising administering a therapeutically effective amount of arimoclomol and a therapeutically effective amount of miglustat to the subject, wherein the subject has an NPC genotype of either Missense/Missense or Missense/Functional Null.

Embodiment 5. Use of a combination of Arimoclomol and miglustat in the manufacture of a medicament for treating or preventing Niemann Pick disease, type C (NPC) in a subject in need thereof, wherein the subject has an NPC genotype of either Missense/Missense or Missense/Functional Null.

Embodiment 5a. Use of a combination of Arimoclomol and miglustat in the manufacture of a medicament for treating Niemann Pick disease, type C (NPC) in a subject in need thereof, wherein the subject has an NPC genotype of either Missense/Missense or Missense/Functional Null.

Embodiment 6. A combination of Arimoclomol and miglustat for use in the treatment or prevention of Niemann Pick disease, type C (NPC) in a subject in need thereof, wherein the subject has an NPC genotype of either Missense/Missense or Missense/Functional Null.

Embodiment 6a. A combination of Arimoclomol and miglustat for use in the treatment of Niemann Pick disease, type C (NPC) in a subject in need thereof, wherein the subject has an NPC genotype of either Missense/Missense or Missense/Functional Null.

Embodiment 7. The method, use, arimoclomol, or combination of any one of the preceding embodiments, wherein the subject does not have an NPC genotype of Functional Null/Functional Null.

Embodiment 8. The method, use, arimoclomol, or combination of any one of the preceding embodiments, wherein the subject has a compound heterozygote NPC genotype.

Embodiment 9. A method of predicting the responsiveness of a subject with Niemann Pick disease, type C (NPC) to treatment with arimoclomol, the method comprising:

a) determining if the subject has an NPC genotype of:
i) Missense/Missense;
ii) Missense/Functional Null; or
iii) Functional Null/Functional Null; and

b) predicting that the subject will respond to treatment with arimoclomol when the subject is determined to have an NPC genotype of Missense/Missense or Missense/Functional Null or predicting that the subject will be less likely to respond to treatment with arimoclomol when the subject is determined to have an NPC genotype of Functional Null/Functional Null.

Embodiment 10. A method of predicting the responsiveness of a subject with Niemann Pick disease, type C (NPC) to treatment with a combination of arimoclomol and miglustat, the method comprising:

a) determining if the subject has an NPC genotype of:
i) Missense/Missense;
ii) Missense/Functional Null; or
iii) Functional Null/Functional Null; and

b) predicting that the subject will respond to treatment with a combination of arimoclomol and miglustat when the subject is determined to have an NPC genotype of Missense/Missense or Missense/Functional Null or predicting that the subject will be less likely to respond to treatment with a combination of arimoclomol and miglustat when the subject is determined to have an NPC genotype of Functional Null/Functional Null.

Embodiment 11. A method of identifying a subject with Niemann Pick disease, type C (NPC) who is likely to be responsive to treatment with arimoclomol, the method comprising:

a) determining if the subject has an NPC genotype of:
i) Missense/Missense;
ii) Missense/Functional Null; or
iii) Functional Null/Functional Null; and

b) identifying the subject as being likely to respond to treatment with arimoclomol when the subject is determined to have an NPC genotype of Missense/Missense or Missense/Functional Null or identifying the subject as less likely to respond to treatment with arimoclomol when the subject is determined to have an NPC genotype of Functional Null/Functional Null.

Embodiment 12. A method of identifying a subject with Niemann Pick disease, type C (NPC) who is likely to be responsive to treatment with a combination of arimoclomol and miglustat, the method comprising:

a) determining if the subject has an NPC genotype of:
i) Missense/Missense;
ii) Missense/Functional Null; or
iii) Functional Null/Functional Null; and

b) identifying the subject as being likely to respond to treatment with a combination of arimoclomol and miglustat when the subject is determined to have an NPC genotype of Missense/Missense or Missense/Functional Null or identifying the subject as less likely to respond to treatment with a combination arimoclomol and miglustat when the subject is determined to have an NPC genotype of Functional Null/Functional Null.

Embodiment 13. A method of treating or preventing Niemann Pick disease, type C (NPC) in a subject in need thereof, the method comprising:

a) determining if the subject has an NPC genotype of:
i) Missense/Missense;
ii) Missense/Functional Null; or
iii) Functional Null/Functional Null; and

b) administering a therapeutically effective amount of arimoclomol to the subject when the subject is determined to have an NPC genotype of Missense/Missense or Missense/Functional Null.

Embodiment 13a. A method of treating Niemann Pick disease, type C (NPC) in a subject in need thereof, the method comprising:

a) determining if the subject has an NPC genotype of:
i) Missense/Missense;
ii) Missense/Functional Null; or
iii) Functional Null/Functional Null; and

b) administering a therapeutically effective amount of arimoclomol to the subject when the subject is determined to have an NPC genotype of Missense/Missense or Missense/Functional Null.

Embodiment 14. A method of treating or preventing Niemann Pick disease, type C (NPC) in a subject in need thereof, the method comprising:

a) determining if the subject has an NPC genotype of:
i) Missense/Missense;
ii) Missense/Functional Null; or
iii) Functional Null/Functional Null; and

b) administering a therapeutically effective amount of arimoclomol and a therapeutically effective amount of miglustat to the subject when the subject is determined to have an NPC genotype of Mi ssense/Missense or Missense/Functional Null.

Embodiment 14a. A method of treating Niemann Pick disease, type C (NPC) in a subject in need thereof, the method comprising:

a) determining if the subject has an NPC genotype of:
i) Missense/Missense;
ii) Missense/Functional Null; or
iii) Functional Null/Functional Null; and

b) administering a therapeutically effective amount of arimoclomol and a therapeutically effective amount of miglustat to the subject when the subject is determined to have an NPC genotype of Mi ssense/Missense or Missense/Functional Null.

Embodiment 15. The method, use, arimoclomol, or combination of any one of the preceding embodiments, wherein the NPC genotype is an NPC1 genotype.

Embodiment 16. The method, use, arimoclomol, or combination of any one of the preceding embodiments, wherein the NPC genotype is an NPC2 genotype.

Embodiment 17. The method, use, arimoclomol, or combination of any one of the preceding embodiments, wherein determining an NPC genotype comprises sequencing the nucleic acid isolated from a biological sample from the subject.

Embodiment 18. A method of treating or preventing Niemann Pick disease, type C (NPC) in a subject in need thereof, the method comprising administering a therapeutically effective amount of arimoclomol to the subject, wherein the subject is identified as having at least one missense mutation in at least one of the two alleles in an NPC gene.

Embodiment 18a. A method of treating Niemann Pick disease, type C (NPC) in a subject in need thereof, the method comprising administering a therapeutically effective amount of arimoclomol to the subject, wherein the subject is identified as having at least one missense mutation in at least one of the two alleles in an NPC gene.

Embodiment 19. Use of Arimoclomol in the manufacture of a medicament for treating or preventing Niemann Pick disease, type C (NPC) in a subject in need thereof, wherein the subject is identified as having at least one missense mutation in at least one of the two alleles in an NPC gene.

Embodiment 19a. Use of Arimoclomol in the manufacture of a medicament for treating Niemann Pick disease, type C (NPC) in a subject in need thereof, wherein the subject is identified as having at least one missense mutation in at least one of the two alleles in an NPC gene.

Embodiment 20. Arimoclomol for use in the treatment or prevention of Niemann Pick disease, type C (NPC) in a subject in need thereof, wherein the subject is identified as having at least one missense mutation in at least one of the two alleles in an NPC gene.

Embodiment 20a. Arimoclomol for use in the treatment of Niemann Pick disease, type C (NPC) in a subject in need thereof, wherein the subject is identified as having at least one missense mutation in at least one of the two alleles in an NPC gene.

Embodiment 21. A method of treating or preventing Niemann Pick disease, type C (NPC) in a subject in need thereof, the method comprising administering a therapeutically effective amount of arimoclomol and a therapeutically effective amount of miglustat to the subject, wherein the subject is identified as having at least one missense mutation in at least one of the two alleles in an NPC gene.

Embodiment 21a. A method of treating Niemann Pick disease, type C (NPC) in a subject in need thereof, the method comprising administering a therapeutically effective amount of arimoclomol and a therapeutically effective amount of miglustat to the subject, wherein the subject is identified as having at least one missense mutation in at least one of the two alleles in an NPC gene.

Embodiment 22. Use of a combination of Arimoclomol and miglustat in the manufacture of a medicament for treating or preventing Niemann Pick disease, type C (NPC) in a subject in need thereof, wherein the subject is identified as having at least one missense mutation in at least one of the two alleles in an NPC gene.

Embodiment 22a. Use of a combination of Arimoclomol and miglustat in the manufacture of a medicament for treating or preventing Niemann Pick disease, type C (NPC) in a subject in need thereof, wherein the subject is identified as having at least one missense mutation in at least one of the two alleles in an NPC gene.

Embodiment 23. A combination of Arimoclomol and miglustat for use in the treatment or prevention of Niemann Pick disease, type C (NPC) in a subject in need thereof, wherein the subject is identified as having at least one missense mutation in at least one of the two alleles in an NPC gene.

Embodiment 23a. A combination of Arimoclomol and miglustat for use in the treatment of Niemann Pick disease, type C (NPC) in a subject in need thereof, wherein the subject is identified as having at least one missense mutation in at least one of the two alleles in an NPC gene.

Embodiment 24. A method of predicting the responsiveness of a subject with Niemann Pick disease, type C (NPC) to treatment with arimoclomol, the method comprising:

a) determining the presence or absence of at least one missense mutation in at least one allele of an NPC gene; and b) predicting that the subject will respond to treatment with arimoclomol when the presence of at least one missense mutation in at least one allele of an NPC gene is determined.

Embodiment 25. A method of predicting the responsiveness of a subject with Niemann Pick disease, type C (NPC) to treatment with a combination of arimoclomol and miglustat, the method comprising:

a) determining the presence or absence of at least one missense mutation in at least one allele of an NPC gene; and;

b) predicting that the subject will respond to treatment with a combination of arimoclomol and miglustat when the presence of at least one missense mutation in at least one allele of an NPC gene is determined.

Embodiment 26. A method of identifying a subject with Niemann Pick disease, type C (NPC) who will be responsive to treatment with arimoclomol, the method comprising:

a) determining the presence or absence of at least one missense mutation in at least one allele of an NPC gene; and;

b) identifying that the subject will be responsive to treatment with arimoclomol when the presence of at least one missense mutation in at least one allele of an NPC gene is determined.

Embodiment 27. A method of identifying a subject with Niemann Pick disease, type C (NPC) who will be responsive to treatment with a combination of arimoclomol and miglustat, the method comprising:

a) determining the presence or absence of at least one missense mutation in at least one allele of an NPC gene; and;

b) identifying that the subject will be responsive to treatment with a combination of arimoclomol and miglustat when the presence of at least one missense mutation in at least one allele of an NPC gene is determined.

Embodiment 28. A method of treating or preventing Niemann Pick disease, type C (NPC) in a subject in need thereof, the method comprising:

a) determining the presence or absence of at least one missense mutation in at least one allele of an NPC gene; and

b) administering a therapeutically effective amount of arimoclomol to the subject when the presence of at least one missense mutation in at least one allele of an NPC gene is determined.

Embodiment 28a. A method of treating Niemann Pick disease, type C (NPC) in a subject in need thereof, the method comprising:

a) determining the presence or absence of at least one missense mutation in at least one allele of an NPC gene; and

b) administering a therapeutically effective amount of arimoclomol to the subject when the presence of at least one missense mutation in at least one allele of an NPC gene is determined.

Embodiment 29. A method of treating or preventing Niemann Pick disease, type C (NPC) in a subject in need thereof, the method comprising:

a) determining the presence or absence of at least one missense mutation in at least one allele of an NPC gene; and

b) administering a therapeutically effective amount of arimoclomol and a therapeutically effective amount of miglustat to the subject when the presence of at least one missense mutation in at least one allele of an NPC gene is determined.

Embodiment 29a. A method of treating Niemann Pick disease, type C (NPC) in a subject in need thereof, the method comprising:

a) determining the presence or absence of at least one missense mutation in at least one allele of an NPC gene; and

b) administering a therapeutically effective amount of arimoclomol and a therapeutically effective amount of miglustat to the subject when the presence of at least one missense mutation in at least one allele of an NPC gene is determined.

Embodiment 30. The method, use, arimoclomol, or combination of any one of the preceding embodiments, wherein the subject is identified as having at least one missense mutation in each of the two alleles of the NPC gene.

Embodiment 31. The method, use, arimoclomol, or combination of any one of the preceding embodiments, wherein the subject is identified as having at least one missense mutation in one allele of the NPC gene and a functional null mutation in the other allele of the NPC gene.

Embodiment 32. The method, use, arimoclomol, or combination of any one of the preceding embodiments, wherein the subject is identified as having a functional null mutation in at most one allele of the two alleles of the NPC gene.

Embodiment 33. The method, use, arimoclomol, or combination of any one of the preceding embodiments, wherein the subject is identified as being a compound heterozygote for the NPC gene.

Embodiment 34. The method, use, arimoclomol, or combination of any one of the preceding embodiments, wherein the NPC gene is NPCJ.

Embodiment 35. The method, use, arimoclomol, or combination of any one of the preceding embodiments, wherein the NPC gene is NPC2.

Embodiment 36. The method, use, arimoclomol, or combination of any one of the preceding embodiments, wherein determining the presence or absence of at least one missense mutation in at least one allele of the NPC gene comprises sequencing nucleic acid isolated from a biological sample from the subject.

Embodiment 37. The method, use, arimoclomol, or combination of any one of the preceding embodiments, wherein a functional null mutation is a mutation that results in a truncated and defective NPC protein being produced from the allele that has the functional null mutation.

Embodiment 38. The method, use, arimoclomol, or combination of any one of the preceding embodiments, wherein a functional null mutation is a frameshift mutation, an aberrant splicing mutation, or a premature stop codon mutation.

Embodiment 39. The method, use, arimoclomol, or combination of any one of the preceding embodiments, wherein the functional null mutation is a frameshift mutation, and wherein the frameshift mutation is selected from Q991fs, D508fs, H641fs, A1108fs, L1204fs, Q119fs, P733fs, L773fs, K822fs, I962fs, A1192fs, and L1248fs.

Embodiment 40. The method, use, arimoclomol, or combination of any one of the preceding embodiments, wherein the functional null mutation is Q991fs.

Embodiment 41. The method, use, arimoclomol, or combination of any one of the preceding embodiments, wherein the functional null mutation is an aberrant splicing mutation, and wherein the aberrant splicing mutation is selected from R518Q/sp, V20sp, G319sp, F1199sp1, and F1199sp2

Embodiment 42. The method, use, arimoclomol, or combination of any one of the preceding embodiments, wherein the functional null mutation is a premature stop codon mutation, wherein the premature stop codon mutation is selected from R116*, L860*, R934*, and E1188*.

Embodiment 43. The method, use, arimoclomol, or combination of any one of the preceding embodiments, wherein the missense mutation results in a single amino-acid change.

Embodiment 44. The method, use, arimoclomol, or combination of any one of the preceding embodiments, wherein the missense mutation results in a substitution, a deletion, an insertion, a duplication, or any combination thereof.

Embodiment 45. The method, use, arimoclomol, or combination of any one of the preceding embodiments, wherein the missense mutation is selected from I1061T, P1007A, S940L, R1186H, G886V, R978C, P1007L, L472P, R518W, G910S, A927V, S954L, G992W, S1004P, T1036M, N1156S, V1165M, N222S, G248V, S357L, R404W, E451K, Y677N, E718D, S734I, G765V, H897Q, A926V, N968S, P981L, M1001V, P1007R, H1016L, T1036A, A1054T, R1059Q, V1078I, F1079L, F1087L, V1141G, M1142T, A1151T, V1155G, and T1205K.

Embodiment 46. The method, use, arimoclomol, or combination of any one of the preceding embodiments, wherein the missense mutation is selected from I1061T, P1007A, M1142T, N1156S, R1186H, S734I, S940L, and S954L.

Embodiment 47. The method, use, arimoclomol, or combination of any one of the preceding embodiments, wherein the missense mutation is selected from I1061T and P1007A

Embodiment 48. The method, use, arimoclomol, or combination of any one of the preceding embodiments, wherein the missense mutation is selected from R978C and N222S.

Embodiment 49. The method, use, arimoclomol, or combination of any one of the preceding embodiments, wherein the missense mutation is an ER type missense mutation.

Embodiment 50. The method, use, arimoclomol, or combination of any one of the preceding embodiments, wherein the ER type missense mutation results in production of an NPC protein this is misfolded, retained at the endoplasmic reticulum (ER) and subsequently targeted for degradation

Embodiment 51. The method, use, arimoclomol, or combination of any one of the preceding embodiments, wherein the ER type missense mutation is selected from the group consisting of I1061T, M1142T, N1156S and R1186H.

Embodiment 52. The method, use, arimoclomol, or combination of any one of the preceding embodiments, wherein the subject has an NPC1 genotype selected from the group consisting of I1061T/E1188*, I1061T/A1151T, I1061T/Q119fs, I1061T/I962fs, T1036M/ I1061T, I1061T/V1141G, N968S/R1186H, N1156S/F1199sp2, Q991fs/I1061T, H1016L/ I1061T, I1061T/A1192fs, I1061T /N1156S,R1186H/R1186H,P1007A /R1186H, and I1061T /D508fs. 26.

Embodiment 53. The method, use, arimoclomol, or combination of any one of the preceding embodiments, wherein the administration provides sustained benefit of arimoclomol over a two-year period.

Embodiment 54. The method, use, arimoclomol, or combination of any one of the preceding embodiments, wherein arimoclomol is administered for about two years or more.

Embodiment 55. The method, use, arimoclomol, or combination of any one of the preceding embodiments, wherein arimoclomol is administered for greater than two years.

Embodiment 56. The method, use, arimoclomol, or combination of any one of the preceding embodiments, wherein the method comprises early treatment initiation with arimoclomol.

Embodiment 57. The method, use, arimoclomol, or combination of any one of the preceding embodiments, wherein the disease course is modified.

Embodiment 58. The method, use, arimoclomol, or combination of any one of the preceding embodiments, wherein the subject is about four years or older.

Embodiment 59. The method, use, arimoclomol, or combination of any one of the preceding embodiments, wherein said administration provides sustained benefit of arimoclomol over a two-year period, wherein the sustained benefit is characterized by the subject exhibiting a 5-domain NPCCSS score increase which is lower compared to observational or placebo over the two-year period.

Embodiment 60. The method, use, arimoclomol, or combination of any one of the preceding embodiments, wherein arimoclomol citrate is administered from about 100 mg/day to about 1000 mg/day.

Embodiment 61. The method, use, arimoclomol, or combination of any one of the preceding embodiments, wherein arimoclomol citrate is administered at about 100 mg/day, about 125 mg/day, about 150 mg/day, about 175 mg/day, about 200 mg/day, about 225 mg/day, about 250 mg/day, about 275 mg/day, about 300 mg/day, about 325 mg/day, about 350 mg/day, about 375 mg/day, about 400 mg/day, about 425 mg/day, about 450 mg/day, about 475 mg/day, about 500 mg/day, about 525 mg/day, about 550 mg/day, about 575 mg/day, about 600 mg/day, about 625 mg/day, about 650 mg/day, about 675 mg/day, or about 700 mg/day.

Embodiment 62. The method, use, arimoclomol, or combination of any one of the preceding embodiments, wherein arimoclomol citrate is administered at about 150 mg/day, about 225 mg/day, about 300 mg/day, about 450 mg/day, or about 600 mg/day.

Embodiment 63. The method, use, arimoclomol, or combination of any one of the preceding embodiments, wherein arimoclomol citrate is administered at about 25 mg, about 50 mg, about 75 mg, about 100 mg, about 125 mg, about 150 mg, about 175 mg, about 200 mg, about 225 mg, about 250 mg, about 275 mg, or about 300 mg.

Embodiment 64. The method, use, arimoclomol, or combination of any one of the preceding embodiments, wherein arimoclomol citrate is administered at about 50 mg, about 75 mg, about 100 mg, about 150 mg, or about 200 mg.

Embodiment 65. The method, use, arimoclomol, or combination of any one of the preceding embodiments, wherein arimoclomol is administered one time, two times, three times, four times, or five times daily.

Embodiment 66. The method, use, arimoclomol, or combination of any one of the preceding embodiments, wherein arimoclomol is administered three time daily.

Embodiment 67. The method, use, arimoclomol, or combination of any one of the preceding embodiments, wherein arimoclomol is administered one, two, three, four, five, six, or seven days a week.

Embodiment 68. The method, use, arimoclomol, or combination of any one of the preceding embodiments, wherein arimoclomol is administered in a dosage adjusted by patient body weight.

Embodiment 69. The method, use, arimoclomol, or combination of any one of the preceding embodiments, wherein the subject has a body weight of about 8 kg to about 15 kg and arimoclomol citrate is administered at a dose of about 50 mg t.i.d. (about 150 mg/day).

Embodiment 70. The method, use, arimoclomol, or combination of any one of the preceding embodiments, wherein the subject has a body weight of about 15 kg to about 22 kg and arimoclomol citrate is administered at a dose of about 75 mg t.i.d. (225 mg/day).

Embodiment 71. The method, use, arimoclomol, or combination of any one of the preceding embodiments, wherein the subject has a body weight of about 22 kg to about 38 kg and arimoclomol citrate is administered at a dose of about 100 mg t.i.d. (about 300 mg/day).

Embodiment 72. The method, use, arimoclomol, or combination of any one of the preceding embodiments, wherein the subject has a body weight of about 38 kg to about 55 kg and arimoclomol citrate is administered at a dose of about 150 mg t.i.d. (about 450 mg/day).

Embodiment 73. The method, use, arimoclomol, or combination of any one of the preceding embodiments, wherein the subject has a body weight of greater than about 55 kg and arimoclomol citrate is administered at a dose of about 200 mg t.i.d. (about 600 mg/day).

Embodiment 74. The method, use, arimoclomol, or combination of any one of the preceding embodiments, wherein arimoclomol is administered orally.

Embodiment 75. The method, use, arimoclomol, or combination of any one of the preceding embodiments, wherein the administration of arimoclomol reduces accumulation of unesterified cholesterol in peripheral blood mononuclear cells.

Embodiment 76. The method, use, arimoclomol, or combination of any one of the preceding embodiments, wherein the administration of arimoclomol reduces accumulation of serum cholestane-triol levels.

Embodiment 77. The method, use, arimoclomol, or combination of any one of the preceding embodiments, wherein the subject is a mammal.

Embodiment 78. The method, use, arimoclomol, or combination of any one of the preceding embodiments, wherein the mammal is a human.

Embodiment 79. The method, use, arimoclomol, or combination of any one of the preceding embodiments, wherein miglustat is administered prior to arimoclomol.

Embodiment 80. The method, use, arimoclomol, or combination of any one of the preceding embodiments, wherein arimoclomol is administered prior to miglustat.

Embodiment 81. The method, use, arimoclomol, or combination of any one of the preceding embodiments, wherein arimoclomol and miglustat are administered simultaneously or sequentially.

Embodiment 82. The method, use, arimoclomol, or combination of any one of the preceding embodiments, wherein miglustat is administered for at least one year prior.

Embodiment 83. The method, use, arimoclomol, or combination of any one of the preceding embodiments, wherein arimoclomol is the citrate salt.

Embodiment 84. The method, use, arimoclomol, or combination of any one of the preceding embodiments, wherein the administration provides sustained benefit of arimoclomol over a three-year period.

Embodiment 85. The method, use, arimoclomol, or combination of any one of the preceding embodiments, wherein arimoclomol is administered for about three years or more.

Embodiment 86. The method, use, arimoclomol, or combination of any one of the preceding embodiments, wherein arimoclomol is administered for greater than three years.

Embodiment 87. The method, use, arimoclomol, or combination of any one of the preceding embodiments, wherein said administration provides sustained benefit of arimoclomol over a three-year period, wherein the sustained benefit is characterized by the subject exhibiting a 5-domain NPCCSS score increase which is lower compared to observational or placebo over the three-year period.

### EXEMPLIFICATION

The following non-limiting example describes a 12-month, prospective, randomized, double-blind, placebo-controlled, phase 2/3 multinational trial performed to test the use of arimoclomol for the treatment of Niemann-Pick disease type C (NPC).

### Methods

Study Participants: the NPC1 genotypes of the subjects enrolled in the study are shown in Table 2. Patients enrolled in the trial were stratified by use of miglustat at baseline. Patients in both strata were randomized 2:1 to receive arimoclomol or placebo.

TABLE 2
NPC1 genotypes of subjects enrolled in study

| NPC1 Allele 1 | NPC1 Allele 2 | |
|---|---|---|
| Amino acid | Amino acid | NPC1 Genotype |
| V20sp | P1007A | Missense/Functional Null |
| Q991fs | P1007A | Missense/Functional Null |
| A927V | S1004P | Missense/Missense |
| S940L | P1007A | Missense/Missense |
| I1061T | E1188* | Missense/Functional Null |
| A926V | P1007A | Missense/Missense |
| R404W | M1001V | Missense/Missense |
| I1061T | A1151T | Missense/Missense |
| I1061T | Q119fs | Missense/Functional Null |
| I1061T | I962fs | Missense/Functional Null |
| S734I | K822fs | Missense/Functional Null |
| P1007L | P1007L | Missense/Missense |
| T1036M | I1061T | Missense/Missense |
| G765V | P1007A | Missense/Missense |
| I1061T | N1156S | Missense/Missense |
| I1061T | V1141G | Missense/Missense |
| E718D | P1007L | Missense/Missense |
| V1165M | R116* | Missense/Functional Null |
| R1186H | R1186H | Missense/Missense |
| N968S | R1186H | Missense/Missense |
| E451K | G992W | Missense/Missense |
| P1007R | T1205K | Missense/Missense |
| R934* | F1079L | Missense/Functional Null |
| A1108fs | A1108fs | Functional Null/Functional Null |
| N1156S | F1199sp2 | Missense/Functional Null |
| V1165M | F1199sp1 | Missense/Functional Null |
| R518W | H641fs | Missense/Functional Null |
| G248V | L773fs | Missense/Functional Null |
| P1007A | R1186H | Missense/Missense |
| A1054T | H897Q | Missense/Missense |
| P733fs | V1078I | Missense/Functional Null |
| H641fs | S954L | Missense/Functional Null |
| D508fs | I1061T | Missense/Functional Null |
| Q991fs | I1061T | Missense/Functional Null |
| L860* | Q991fs | Functional Null/Functional Null |
| G910S | G910S | Missense/Missense |
| P1007A | L1204fs | Missense/Functional Null |
| P1007A | L1204fs | Missense/Functional Null |
| G886V | R978C | Missense/Missense |
| G886V | R978C | Missense/Missense |
| S357L | S940L | Missense/Missense |
| R518W | G992W | Missense/Missense |
| S940L | S940L | Missense/Missense |
| R518Q | R518Q | Functional Null/Functional Null |
| Y677N | R1059Q | Missense/Missense |
| T1036A | V1155G | Missense/Missense |
| H1016L | I1061T | Missense/Missense |
| P981L | L1248fs | Missense/Functional Null |
| L472P | L472P | Missense/Missense |
| I1061T | A1192fs | Missense/Functional Null |

Study Procedures: the screening visit (visit 1) included a baseline assessment, randomization assignment, and pharmacokinetic (PK) assessment. The first dose of arimoclomol or placebo was given within 1 week of randomization. Safety assessments were performed at visit 2 (7-14 days after start of treatment) and then every 3 months during the blinded period (visits 3-6). Monthly telephone follow-ups were performed to evaluate safety, confirm patients' weights, and assess treatment compliance.

Routine clinical care was maintained throughout the trial (including administration of miglustat). Each participant was randomized to receive arimoclomol or placebo three times daily. Arimoclomol was administered orally or by feeding tube at 93-372 mg/day based on the patient's body weight up to the estimated equivalence of 372 mg/day for adults (body weight >55 kg) or of 124 mg three times a day.

During the 12-month treatment phase, efficacy assessments for the primary endpoint, the 5-domain NPCCSS score, and for the non-disease specific Clinical Global Impression—Improvement scale (CGI-I) scores were performed at baseline and after 3, 6, 9, and 12 months of treatment; all other efficacy and biomarker assessments were performed at baseline and after 6 and 12 months of treatment.

### Outcomes

Primary endpoint: The primary endpoint was change from baseline in NPC severity at 12 months as assessed by the 5-domain NPCCSS score, an abbreviated assessment tool originating from the 17 domain NPCCSS score developed by Yanjanin et al., as described herein. The fully validated 5-domain NPCCSS score comprises the domains determined to be most clinically relevant to patients, caregivers, and clinicians: ambulation, cognition, fine motor skills, speech, and swallowing. The total aggregated 5-domain NPCCSS score ranges from 0 to 25, with a higher score indicating more severe clinical impairment.

Subgroup analyses of primary endpoint: NPC is a heterogeneous disease, and patients aged 2 to 18 years present with a large spectrum of disease presentations. The group of children <4 years old includes patients with mild manifestations and patients with aggressive, early fatal disease. As this group is particularly heterogeneous, a subgroup of patients >4 years of age was predefined with the population of study participants.

In the EU, miglustat is indicated for the treatment of progressive neurological manifestations in patients with NPC. However, not all patients are candidates for miglustat treatment, and it is recommended that the benefit of treatment should be evaluated on a regular basis (e.g., every six months). An analysis of the subpopulation of patients receiving miglustat was prespecified to elucidate the effect of arimoclomol in patients on background miglustat treatment. Overall, the subgroups of patients ≥4 years of age, and patients on miglustat treatment, were expected to be more homogeneous with respect to baseline demographics and disease characteristics and therefore more suitable for comparison.

Two post hoc subgroup analyses were also conducted.

In the first post hoc analysis, subjects with double functional null mutations in NPC1 (NPC1 genotype of Functional Null/Functional Null) were excluded.

In the second post hoc analysis, only subjects with annual severity increment score (ASIS) 0.5-2.0 were included. Cortina-Borja et al. suggests that by applying differential ASIS cut-off points of 0.5-2.0, which excludes the very mild and very severe patients, trial cohorts may be stratified to obtain a more homogeneous patient population that, if untreated, would change in clinical score within the typical period of a clinical trial.

Secondary endpoints: A secondary endpoint was responder analysis of Clinical Global Impression-Improvement scale (CGI-I) scores (responder defined as stable or improved) at 12 months compared with baseline. Other secondary endpoints were: responder analysis of 5-domain NPCCSS score (defined as stable or improved) at month 12 versus baseline; time to worsening on 5-domain NPCCSS score (defined as the time until the patient worsened by 2 points vs baseline); proportion of patients worsening on 5-domain NPCCSS score at 6 and 12 months by 2 points on the 5-domain NPCCSS score; and change in 17-domain NPCCSS score (excluding hearing domains) at 12 months. Additional secondary endpoints included: change from baseline in scale for assessment and rating of ataxia (SARA) score at 6 and 12 months; change in the nine-hole peg test (9-HPT) result at 6 and 12 months; change in health-related quality of life (HRQoL) as measured by the 5-dimension 3-level EuroQol questionnaire, youth version (EQ-5D-3L Y proxy) at 6 and 12 months; change in individual 5-domain NPCCSS score at month 12; and NPC clinical database (NPC-cdb) score changes from baseline at trial time points. The NPC-cdb score aims to reflect clinical status; an increase in score indicates a reduction in the patient's abilities. The score was calculated as defined by Stampfer et al. (see Stampfer M, Theiss S, Amraoui Y, Jiang X, Keller S, Ory D. Niemann-Pick disease type C clinical database: cognitive and coordination deficits are early disease indicators. Orphanet J Rare Dis. 2013; 8:35 doi: 10.1186/750-72-8-35, incorporated herein by reference in its entirety).

The skilled artisan would readily appreciate that the SARA score, the 9-HPT, the HRQoL and the NPC-cdb are standardized, widely used clinical analysis techniques used to determine NPC progression in subjects.

CGI-I: the CGI-I was originally developed as a research rating tool to assess psychiatric diseases (see Busner J, Targum SD. The clinical global impressions scale: applying a research tool in clinical practice. Psychiatry (Edgmont). 2007;4:28-37, incorporated herein by reference in its entirety). It provides the clinician's impression of improvement (or worsening) of a person's condition at the current visit compared with baseline on a 7-point scale ranging from 1 (very much improved) to 7 (very much worse). In the present trial, CGI-I was performed after clinical examination and patient interviews; the same investigator was instructed to perform CGI-I assessments throughout the trial for a given patient.

SARA: The SARA includes eight items reflecting neurological manifestations of cerebellar ataxia and provides a direct and simple description of motor function. The total score of the eight items ranges from 0 (normal) to 40 (not able to perform any of the test items). See Schmitz-Hubsch T, du Montcel ST, Baliko L, et al. Scale for the assessment and rating of ataxia: development of a new clinical scale. Neurology. 2006; 66:1717-20, incorporated herein by reference in its entirety.

9-HPT: The 9-HPT is a direct and simple measurement of fine motor coordination, hand/eye coordination, and the ability to follow a simple direction, measured in seconds for each hand. The test is not applicable for children under 4 years of age. See Poole J L, Burtner PA, Torres TA, et al. Measuring dexterity in children using the Nine-hole Peg Test. J Hand Ther. 2005; 18:348-51.

EQ-5D-3L Y proxy: The HRQoL of individuals in the trial was measured using the child-friendly version of the EQ-5D-3L Y proxy questionnaire. The questionnaire was completed by the patient's caregiver as a proxy for the individual.

Quantification of Biomarkers: Cholestane-triol in serum, unesterified cholesterol, and HSP70 levels in peripheral blood mononuclear cells (PBMCs) were measured as described in Mengel E, Bembi B, Del Toro M, et al. Clinical disease progression and biomarkers in Niemann—Pick disease type C: a prospective cohort study. Orphanet J Rare Dis. 2020; 15:328, incorporated herein by reference in its entirety.

### Results

The 50 subjects in the study were randomized (26 females; 24 males) from 14 sites in nine countries. Thirty-four patients received arimoclomol and 16 received placebo.

The proportion of patients completing 12 months of randomized treatment was 79.4% in the arimoclomol group and 93.8% in the placebo group. In the arimoclomol group, reasons for withdrawal included adverse events (n=3), withdrawal of consent (n=1), fast disease progression (early escape clause; n=2), and death from NPC progression (n=1). In the placebo group, one patient withdrew after one day owing to worsening of epilepsy (considered part of disease progression).

Baseline disease characteristics and demographics of patients are described in Table 3.

TABLE 3
Baseline characteristics and demographics of subjects in study

| | Arimoclomol | | Placebo | | Total | |
|---|---|---|---|---|---|---|
| | (n = 34) | | (n = 16) | | (n = 50) | |
| Age, years (mean [SD]) | 11.5 | (5.4) | 10.2 | (4.1) | 11.1 | (5.0) |
| Sex | | | | | | |
| Male | 17 | (50.0) | 7 | (43.8) | 24 | (48.0) |
| Female | 17 | (50.0) | 9 | (56.3) | 26 | (52.0) |
| Race | | | | | | |
| White | 32 | (94.1) | 13 | (81.3) | 45 | (90.0) |
| Asian | 1 | (2.9) | 1 | (6.3) | 2 | (4.0) |
| Native Hawaiian or | 0 | | 1 | (6.3) | 1 | (2.0) |
| other Pacific Islander | | | | | | |
| Other | 1 | (2.9) | 1 | (6.3) | 2 | (4.0) |
| BMI (kg/m²) | 18.72 | (4.15) | 19.46 | (3.33) | 18.95 | (3.89) |
| Age at diagnosis of first | 5.05 | (3.43) | 5.22 | (3.87) | 5.10 | (3.54) |
| neurological symptom | | | | | | |
| (years), mean (SD) | | | | | | |
| Age at first NPC | | | | | | |
| symptom (years), n % | | | | | | |
| Pre/peri-natal | 1 | (2.9) | 0 | | 1 | (2.0) |
| (<3 months) | | | | | | |
| Early-infantile | 5 | (14.7) | 3 | (18.8) | 8 | (16.0) |
| (3 months to <2 years) | | | | | | |
| Late-infantile | 17 | (50.0) | 7 | (43.8) | 24 | (48.0) |
| (2 to <6 years) | | | | | | |
| Juvenile (6 to 15 years) | 11 | (32.4) | 6 | (37.5) | 17 | (34.0) |
| Adolescent/adult | 0 | | 0 | | 0 | |
| (>15 years) | | | | | | |
| Time since first NPC | | | | | | |
| symptom (years) | | | | | | |
| Mean (SD) | 7.61 | (4.54) | 8.07 | (3.75) | 7.76 | (4.27) |
| Median | 6.15 | | 8.10 | | 7.00 | |
| Range | 0.4-16.6 | | 2.0-14.8 | | 0.4-16.6 | |
| Time since NPC | | | | | | |
| diagnosis (years) | | | | | | |
| Mean (SD) | 5.59 | (4.36) | 5.11 | (4.14) | 5.43 | (4.25) |
| Median | 4.10 | | 3.00 | | 3.90 | |
| Range | 0.1-15.1 | | 0.8-14.2 | | 0.1-15.1 | |
| Treated with miglustat | | | | | | |
| Yes | 26 | (76.5) | 13 | (81.3) | 39 | (78.0) |
| History of seizure or | 12 | (35.3) | 2 | (12.5) | 14 | (28.0) |
| epilepsy, n (%) | | | | | | |
| Baseline 5-domain | | | | | | |
| NPCCSS score | | | | | | |
| Mean (SD) | 12.1 | (6.9) | 9.4 | (6.4) | 11.2 | (6.8) |
| Median | 11.5 | | 8.0 | | 10.5 | |
| Range | 1.0-24.0 | | 0.0-24.0 | | 0.0-24.0 | |
| Baseline 5-domain | | | | | | |
| NPCCSS score; | | | | | | |
| individual domain | | | | | | |
| scores | | | | | | |
| Ambulation score; | 2.5 | (1.6) | 2.2 | (1.6) | 2.4 | (1.6) |
| mean (SD) | | | | | | |
| Speech score; | 2.1 | (1.6) | 1.6 | (1.2) | 2.0 | (1.5) |
| mean (SD) | | | | | | |
| Swallow score; mean | 1.9 | (1.7) | 1.3 | (1.7) | 1.7 | (1.7) |
| (SD) | | | | | | |
| Fine motor skills score; | 2.8 | (1.8) | 1.9 | (1.8) | 2.5 | (1.9) |
| mean (SD) | | | | | | |
| Cognition score; mean | 2.8 | (1.3) | 2.5 | (1.5) | 2.7 | (1.3) |
| (SD) | | | | | | |
| Baseline full-scale | 21.1 | (11.5) | 17.2 | (11.3) | 19.8 | (11.5) |
| NPCCSS score, except | | | | | | |
| hearing domains, mean | | | | | | |
| (SD) | | | | | | |
| Baseline NPC-cdb, | 46.5 | (24.0) | 39.2 | (28.6) | 44.1 | (25.6) |
| mean (SD) | | | | | | |

All subjects had a diagnosis of NPC with mutations in both NPC1 alleles. Most patients (n=39/50) were receiving miglustat as part of routine clinical care. Baseline mean 5-domain NPCCSS score, 17-domain NPCCSS score (excluding hearing domains), and NPC-cdb scores were higher in the arimoclomol group than in the placebo group (see Table 3). Subgroup baseline characteristics are summarized in Table 4.

TABLE 4
Baseline disease characteristics and demographics by subgroup
(FAS per subgroup).

| | Arimoclomol | | Placebo | | Total | |
|---|---|---|---|---|---|---|
| Receiving concomitant miglustat | 26 | | 13 | | 39 | |
| Age (years) | | | | | | |
| Mean (SD) | 12.8 | (4.7) | 9.1 | (3.6) | 11.6 | (4.7) |
| Baseline 5-domain NPCCSS score | | | | | | |
| Mean (SD) | 11.7 | (7.2) | 9.6 | (7.1) | 11.0 | (7.1) |
| Median (range) | 10.5 | (1.0-24.0) | 8.0 | (0-24.0) | 10.0 | (0-24.0) |
| Age at first neurological symptom | | | | | | |
| (years) | | | | | | |
| Mean (SD) | 5.25 | (3.34) | 4.04 | (3.20) | 4.85 | (3.31) |
| Patients with double functional null | 0 | | 0 | | 0 | |
| mutation (Functional Null/Functional | | | | | | |
| Null), n | | | | | | |
| Not receiving concomitant miglustat, n | 8 | | 3 | | 11 | |
| Age (years) | | | | | | |
| Mean (SD) | 7.0 | (5.4) | 15.0 | (1.7) | 9.2 | (5.9) |
| Baseline 5-domain NPCCSS score | | | | | | |
| Mean (SD) | 13.3 | (6.1) | 8.7 | (2.1) | 12.0 | (5.6) |
| Median (range) | 14.0 | (2.0-20.0) | 8.0 | (7.0-11.0) | 11.0 | (2.0-20.0) |
| Age at first neurological symptom | | | | | | |
| (years) | | | | | | |
| Mean (SD) | 4.39 | (3.87) | 10.33 | (1.53) | 6.01 | (4.32) |
| Patients with double functional null | 3 | | 0 | | 3 | |
| mutation, n | | | | | | |
| Age ≥4 years, n | 30 | | 14 | | 44 | |
| Age (years) | | | | | | |
| Mean (SD) | 12.7 | (4.5) | 11.2 | (3.2) | 12.2 | (4.2) |
| Baseline 5-domain NPCCSS score | | | | | | |
| Mean (SD) | 12.0 | (6.9) | 10.3 | (6.4) | 12.2 | (4.2) |
| Median (range) | 13.5 | (4.0-19.0) | 11.0 | (7.0-16.0) | 12.5 | (4.0-19.0) |
| Age at first neurological symptom | | | | | | |
| (years) | | | | | | |
| Mean (SD) | 5.57 | (3.29) | 5.74 | (3.86) | 5.62 | (3.44) |
| Patients with double functional null | 0 | | 0 | | 0 | |
| mutation, n | | | | | | |
| Age <4 years, n | 4 | | 2 | | 6 | |
| Age (years) | | | | | | |
| Mean (SD) | 2.5 | (0.6) | 3.0 | (0.0) | 2.7 | (0.5) |
| Baseline 5-domain NPCCSS score | | | | | | |
| Mean (SD) | 12.5 | (7.9) | 3.5 | (0.7) | 9.5 | (7.7) |
| Median (range) | 14.5 | (2.0-19.0) | 3.5 | (3.0-4.0) | 7.5 | (2.0-19.0) |
| Age at first neurological symptom | | | | | | |
| (years) | | | | | | |
| Mean (SD) | 1.13 | (1.30) | 1.58 | (0.59) | 1.28 | (1.07) |

Three patients, all randomized to the arimoclomol group, had double functional null mutations (see Table 5).

TABLE 5
Genotype analysis of NPC1 mutations of enrolled subjects

| | Arimoclomol | | Placebo | | Total | |
|---|---|---|---|---|---|---|
| | (n = 34 | | (n = 16 | | (n = 50 | |
| | subjects) | | subjects) | | subjects) | |
| Patient genotypes by | | | | | | |
| mutation type, n (%) | | | | | | |
| Double functional | 3 | (8.8) | 0 | (0) | 3 | (6.0) |
| null | | | | | | |
| Double missense | 16 | (47.1) | 11 | (68.8) | 27 | (54.0) |
| Missense/functional | 15 | (44.1) | 5 | (31.2) | 20 | (40.0) |
| null | | | | | | |
| | Arimoclomol | | Placebo | | Total | |
| | (n = 68 | | (n = 32 | | (n = 100 | |
| | alleles) | | alleles) | | alleles) | |
| Frequency of | | | | | | |
| protein mutation | | | | | | |
| types, n alleles % | | | | | | |
| Missense | 47 | (69.1) | 27 | (84.4) | 74 | (74.0) |
| Functional null | 21 | (30.9) | 5 | (15.6) | 26 | (26.0) |
| Frameshift | 12 | (17.6) | 5 | (15.6) | 17 | (17.0) |
| Splicing | 5 | (7.4) | 0 | (0) | 5 | (5.0) |
| Premature stop | 4 | (5.9) | 0 | (0) | 4 | (4.0) |

| | Type | Genotype |
|---|---|---|
| Characteristics of | | |
| double functional | | |
| null mutations | | |
| (arimoclomol group) | | |
| Patient 1 | Frameshift/frameshift | A1108fs/ |
| | | A1108fs |
| Patient 2 | Premature stop/ | L860*/ |
| | frameshift | Q991fs |
| Patient 3 | Splicing/splicing | R518Q/ |
| | | R518Q |

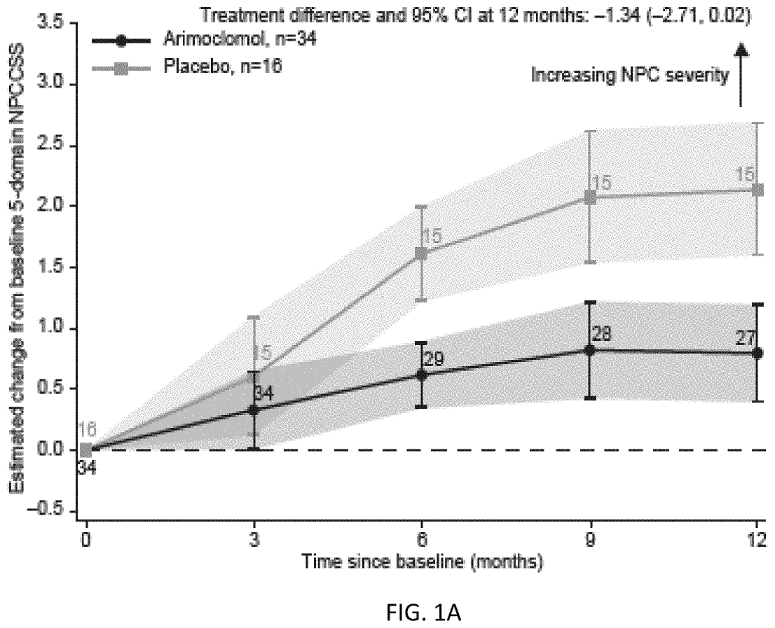

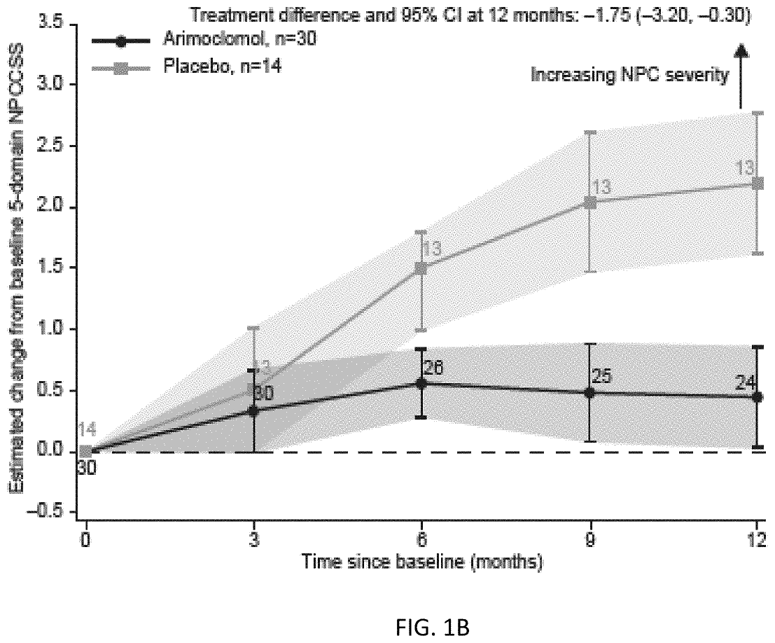

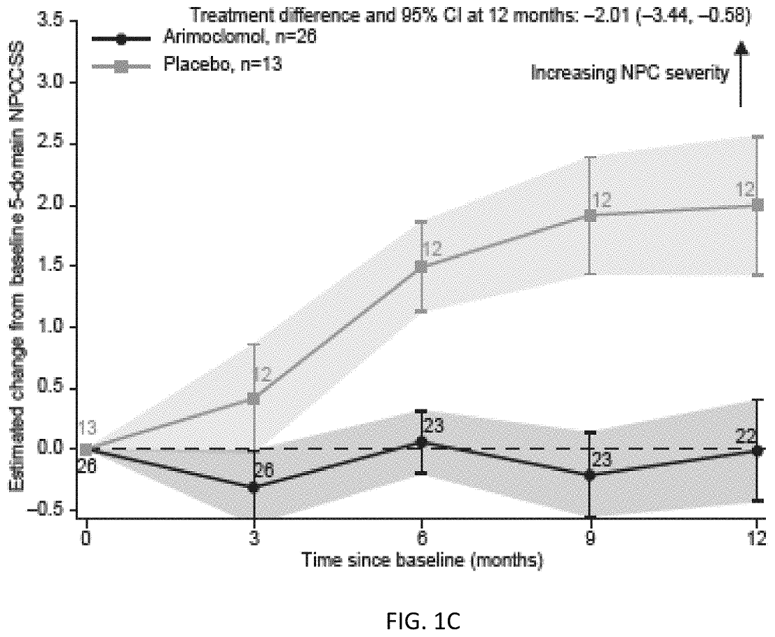

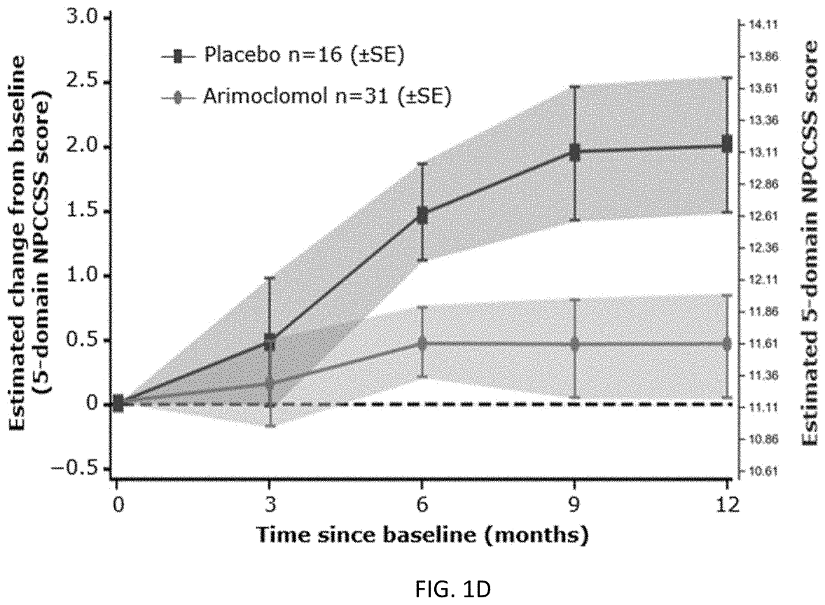

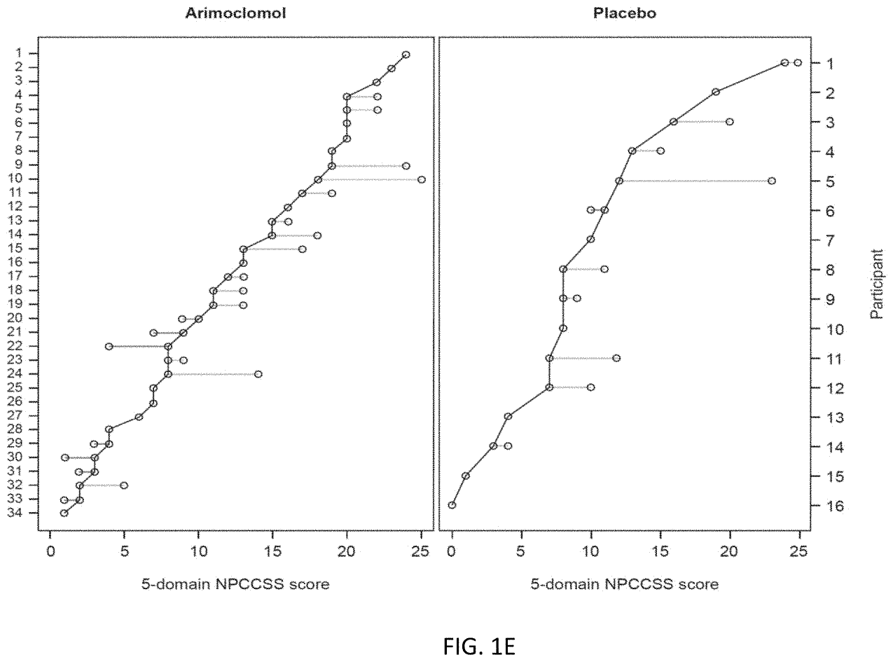

Efficacy Evaluation: For the primary endpoint, at month 12, mean (95% confidence interval [CI]) change on the 5-domain NPCCSS score was 0.80 (−0.01, 1.60) for arimoclomol compared with 2.14 (1.04, 3.24) for placebo, corresponding to a treatment effect in favor of arimoclomol of −1.34 (95% CI: −2.71, 0.02; p=0.0537; see FIG. 1A) and a 63% relative reduction in annual disease progression. Patient-level data for the change in 5-domain NPCCSS score are presented in FIG. 1E.

The primary multilevel modeling for repeated measures (MIVIRM) model was applied on the prespecified subgroup levels with enough patients to substantiate a formal analysis. In the subgroups of patients >4 years old (n=44; see FIG. 1B) and patients concomitantly receiving miglustat (n=39; FIG. 1C), treatment with arimoclomol results in slowed progression of NPC (p<0.05; see Table 6).

TABLE 6
Change in clinical endpoints (full analysis set, except subgroup analyses).

| Change in 5-domain | | | | | Arimoclomol vs | | |
|---|---|---|---|---|---|---|---|
| NPCCSS score from | Arimoclomol | | Placebo | | placebo: Difference | | p |
| baseline at 12 months | (n = 34) | | (n = 16) | | (95% CI) | | value |
| Overall population, n | 34 | (27) | 16 | (15) | | | |
| (n at 12 months) | | | | | | | |
| Mean change (95% CI) | 0.80 | (−0.01, 1.60) | 2.14 | (1.04, 3.24) | −1.34 | (-2.71, 0.02) | 0.0537 |
| Relative reduction in | | | | | 63 | | |
| annual disease progression, | | | | | | | |
| % | | | | | | | |
| Subgroup analyses | | | | | | | |
| Individuals receiving | 26 | (22) | 13 | (12) | | | |
| miglustat, n (n at 12 months) | | | | | | | |
| Mean change (95% CI) | −0.01 | (−0.85, 0.83) | 2.00 | (0.84, 3.15) | −2.01 | (−3.44, −0.58) | 0.0074 |
| Relative reduction in | | | | | 101 | | |
| annual disease progression, | | | | | | | |
| % | | | | | | | |
| Individuals not receiving | 8 | (3) | 3 | (3) | | | |
| miglustat, n (n at 12 months) | | | | | | | |
| Mean change (95% CI) | 4.2 | (1.7, 6.71) | 1.99 | (−1.6, 5.57) | 2.21 | (−2.14, 6.57) | 0.2835 |
| Relative reduction in | | | | | NA | | |
| annual disease progression, | | | | | | | |
| % | | | | | | | |
| Individuals aged ≥4 years, n | 30 | (24) | 14 | (13) | | | |
| (n at 12 months) | | | | | | | |
| Mean change (95% CI) | 0.44 | (−0.40, 1.28) | 2.19 | (1.02, .37) | −1.75 | (−3.20, −0.30) | 0.0190 |
| Relative reduction in | | | | | 80 | | |
| annual disease progression, | | | | | | | |
| % | | | | | | | |
| Individuals aged <4 years, n | 3 | (3) | 2 | (2) | | | |
| (n at 12 months) | | | | | | | |
| Mean change (95% CI) | NC | | NC | | | | |
| Relative reduction in | | | | | NA | | |
| annual disease progression, | | | | | | | |
| % | | | | | | | |
| Responders on 5-domain | 17 | (50.0) | 6 | (37.5) | 12.5 | (−16.6, 41.6) | 0.5456 |
| NPCCSS score at 12 months, | | | | | | | |
| n (%) | | | | | | | |
| Proportion worsening on | 15 | (44.1) | 7 | (43.8) | 0.37 | (−29.1, 29.8) | 1.0000 |
| 5-domain NPCCSS score at | | | | | | | |
| 12 months, n (%) | | | | | | | |
| Time to worsening on | 3.68 | (2.89, 5.95) | 4.29 | (1.94, 6.48) | NA | | 0.8733 |
| 5-domain NPCCSS score, | | | | | | | |
| months (95% CI) | | | | | | | |
| Full NPCCSS score | 25 | | 15 | | | | |
| (excluding hearing domains) | | | | | | | |
| at 12 months, n | | | | | | | |
| Mean (SD) | 1.2 | (2.6) | 2.7 | (5.4) | | | |
| Median (IQR) | 1.0 | (−6.0to6.0) | 0.0 | (−7.0, 13.0) | | | |
| LS mean change from | 1.24 | (−0.35 to 2.84) | 2.80 | (0.74, 4.86) | −1.56 | (−4.18, 1.06) | 0.5726 |
| baseline (95% CI) | | | | | | | |
| Responders on CGI-I at 12 | 20/34 | (58.8) | 9/16 | (56.3) | 2.6 | (−26.8, 32.0) | 1.0000 |
| months, n (%) | | | | | | | |
| NPC-cdb score change from | 1.85 | (−2.16 to 5.86) | 4.88 | (−0.63, 10.39) | −3.03 | (−9.90, 3.85) | 0.3785 |
| baseline to 12 months, LS | | | | | | | |
| mean (95% CI) | | | | | | | |
| SARA score change from | 1.06 | (−0.17 to 2.29) | 0.78 | (−0.90, 2.47) | 0.28 | (−1.82, 2.37) | 0.7899 |
| baseline to 12 months, LS | | | | | | | |
| mean (95% CI) | | | | | | | |
| EQ-5D-3L Y proxy, n (%) | | | | | | | |
| Improved at 12 months | 7/27 | (25.9) | 6/15 | (40.0%) | −14.1 | (−43.9, 15.7) | 0.4880 |
| Worsened at 12 months | 12/27 | (44.4) | 3/15 | (20.0%) | 24.4 | (−3.1, 52.0) | 0.1804 |
| 9-HPT time (s), change from | | | | | | | |
| baseline to 12 months, LS | | | | | | | |
| mean (95% CI) | | | | | | | |
| Dominant hand | −3.29 | (−15.56, 8.98) | −6.49 | (−20.34, 7.37) | 3.20 | (−15.71, 22.12) | 0.7283 |
| Non-dominant hand | 11.68 | (−14.89, 38.25) | 17.59 | (−13.24, 48.42) | −5.91 | (−47.54, 35.72) | 0.7708 |

Based on 5-domain NPCCSS scores, the proportion of responders (stable or improved) was 50.0% and 37.5% in the arimoclomol and placebo groups, respectively (see Table 6). At 12 months, the mean difference in change from baseline in the 17-domain NPCCSS score (excluding hearing domain) and NPC-cdb scores for the arimoclomol versus placebo groups were numerically in favor of arimoclomol (p=0.5726 and p=0.3785, respectively; see Table 6). For the majority of patients (42/50), trial investigators completed baseline CGI-I assessments retrospectively.

To determine whether arimoclomol showed particular efficacy within certain subsets of the subjects in the study, additional post hoc analyses based on ASIS and genotype were performed. When analyzing patients that had a baseline ASIS of 0.5-2.0 (n=21), signal enhancement was observed with a treatment difference of −2.39 in favor of arimoclomol (see Table 7). In addition, when patients that had an NPC1 genotype of Functional Null/Functional Null were excluded from the analysis, there was a dramatically enhanced signal of treatment effect of −1.56 (see Table 7 and FIG. 1D). Thus, these results indicate that arimoclomol treatment is especially efficacious in patients that do not have an NPC1 genotype of Functional Null/Functional Null or that have a baseline ASIS of 0.5-2.0.

TABLE 7
Change in clinical endpoints additional subgroup analyses.

| | | | | | Arimoclomol vs | | |
|---|---|---|---|---|---|---|---|
| | Arimoclomol | | Placebo | | placebo: Difference | | p |
| | (n = 34) | | (n = 16) | | (95% CI) | | value |
| Excluding individuals | 31 | | 16 | | | | |
| with double null | | | | | | | |
| functionl mutations, | | | | | | | |
| n | | | | | | | |
| Mean change | 0.47 | (−0.34, 1.28) | 2.03 | (0.96, 3.09) | −1.56 | (−2.90, −0.21) | 0.0242 |
| (95% CI) | | | | | | | |
| Relative reduction in | | | | | 77 | | |
| annual disease | | | | | | | |
| progression, % | | | | | | | |
| ASIS within 0.5 and | 13 | | 8 | | | | |
| 2, n | | | | | | | |
| Mean change | 0.19 | (−1.26, 1.63) | 2.58 | (0.65, 4.52) | −2.39 | (−4.83, 0.04) | 0.0536 |

| | |
|---|---|
| (95% CI) | |
| Relative reduction in | 93 |
| annual disease | |
| progression, % | |

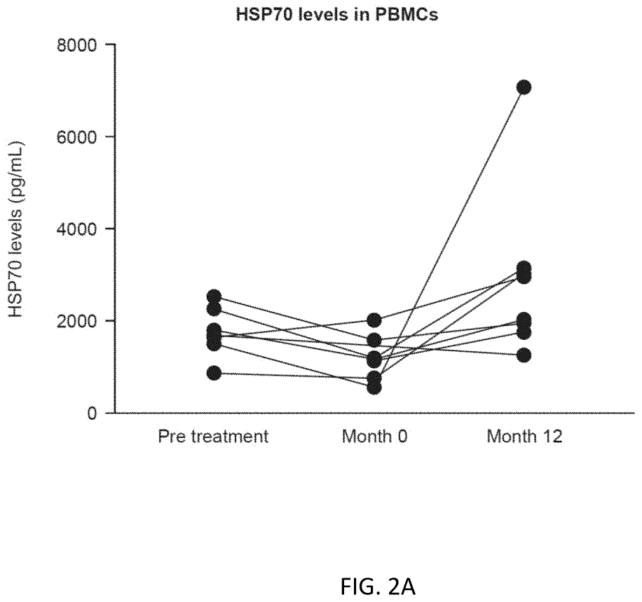

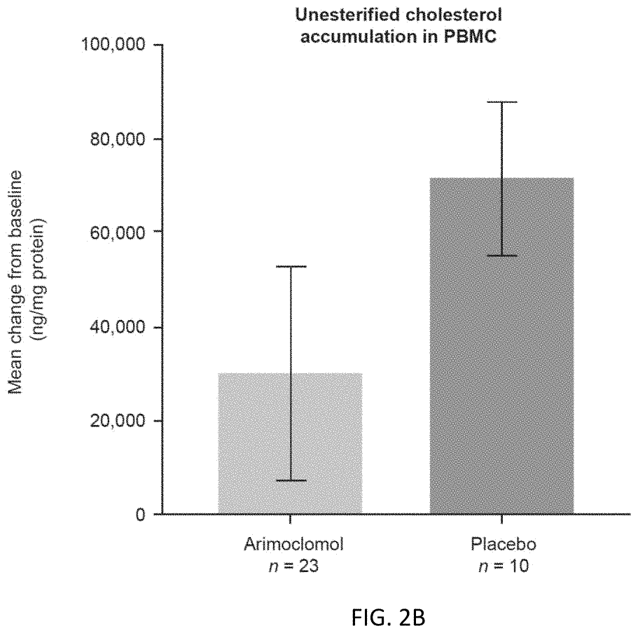

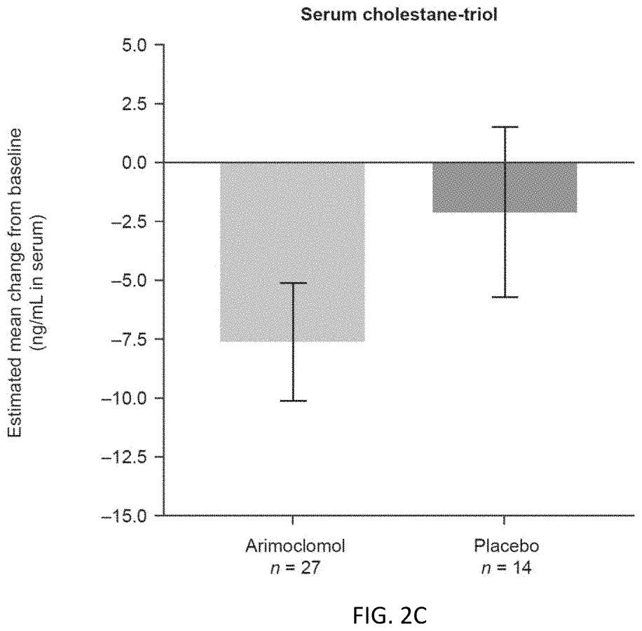

An increase in HSP70 level was observed in response to 12 months of treatment with arimoclomol (n=11; mean [standard deviation] change from baseline 1778.98 [1835.56] pg/mL; p=0.001; see FIG. 2A). Unesterified cholesterol levels in PBMCs increased from baseline to month 12 in both placebo- and arimoclomol-treated patients. The accumulation of unesterified cholesterol was numerically less in arimoclomol- than placebo-treated patients (mean treatment difference [standard error (SE)]−44.44 [25.83] μg/mg protein; p=0.096; see FIG. 2B). A numerical decrease in serum cholestane-triol level was observed in the arimoclomol group relative to the placebo group at 12 months (mean treatment difference [SE] −5.50 [4.46] ng/mL; p=0.225; see FIG. 2C).

Safety Evaluation: In total, 88.2% (30/34) of patients in the arimoclomol group and 75.0% (12/16) in the placebo group had TEAEs. The most common TEAE in both treatment groups was vomiting (arimoclomol: 8/34, 23.5%; placebo: 4/16, 25.0%). Upper respiratory tract infection and decreased weight occurred more frequently with arimoclomol versus placebo, whereas nasopharyngitis, respiratory tract infection, and epilepsy were reported more often with placebo versus arimoclomol. Serious TEAEs occurred in 14.7% (5/34) of patients receiving arimoclomol compared with 31.3% (5/16) of those receiving placebo. All serious TEAEs except those leading to discontinuation from the trial were considered related to NPC. One patient died owing to cardiopulmonary arrest assessed as being related to NPC and not to treatment with arimoclomol. Three patients in the arimoclomol group (8.8%) had four TEAEs that led to trial drug discontinuation. These included two events of urticaria and one of angioedema (all classed as serious and as probably related to investigational product), and one of increased blood creatinine level twice the patient's baseline value (assessed as being related to investigational product). Six patients in the arimoclomol group had an increase in serum creatinine level over 1.5 times their baseline values; for two of these patients (both in the arimoclomol group), levels were over twice baseline values. For one of these patients, the increase occurred during treatment with rescue medication; for the other the elevation was reported as a TEAE and the patients discontinued involvement in the trial, in line with the protocol. None of the patients had any other indications of affected kidney function. For all patients, the creatinine level started to rise at the first measurement after exposure to arimoclomol and was seen to peak before the end of the trial. There were no significant changes in vital signs, electrocardiograms, or other laboratory values during the trial.

Without wishing to be bound by theory, the results of the placebo-controlled phase 2/3, 12-month clinical trial of arimoclomol in NPC described above demonstrates that arimoclomol was well tolerated with clinically meaningful benefit of arimoclomol versus placebo observed in the tested subjects. Significant reduction of disease progression was observed in patients over four years of age and in those concomitantly treated with miglustat. Moreover, arimoclomol treatment demonstrated increased efficacy in subjects that did not have an NPC1 genotype of Functional Null/Functional Null (i.e. subjects with an NPC1 genotype of either Missense/Missense or Missense/Functional Null).

OPEN-LABEL PHASE 2/3 EXTENSION IN NIEMANN-PICK DISEASE TYPE C

The following non-limiting example describes 12-month interim data and 24 month data from an open-label extension (OLE) of the phase 2/3 study of arimoclomol, performed to test the sustained benefit of arimoclomol for the treatment of Niemann-Pick disease type C (NPC).

Results from the 12-month open-label extension of the phase 2/3 randomized placebo-controlled trial (CT-ORZY-NPC-002) demonstrate sustained benefit of arimoclomol over a two-year period and further evidence of its efficacy and safety profile. Forty-one patients completed the 12-month double-blinded part of the CT-ORZY-NPC-002 trial and continued into the open-label extension, where all patients received arimoclomol treatment.

Patients who switched from placebo to arimoclomol treatment experienced similar reduction of disease progression as observed earlier in those patients randomized to arimoclomol treatment in the placebo-controlled trial as measured by the 5-domain NPCCSS (0.23 progression in the open label extension vs 2.0 progression in the placebo-controlled trial).

Patients who received arimoclomol for a total of two years showed greater progression in the open-label extension compared to the placebo-controlled part. This was mainly due to patients under 4 years with continued aggressive disease course. In the predefined subgroups of patients 4 years and older and patients receiving miglustat as part of their routine clinical care, early treatment initiation with arimoclomol resulted in greater benefit than delayed start of treatment, indicating that the disease course was modified by the treatment. Arimoclomol was safe and well-tolerated over 24 months.

The following non-limiting example describes 24-month interim data and 36 month data (M36) from an open-label extension (OLE) of the phase 2/3 study of arimoclomol, performed to test the sustained benefit of arimoclomol for the treatment of Niemann-Pick disease type C (NPC).

All OLE patients received arimoclomol; routine clinical care, including miglustat, was maintained. The primary endpoint was change in disease severity based on 5-domain NPC Clinical Severity Scale (5D-NPCCSS) scores from baseline to Months 18, 24, 30 and 36. Subgroups comprised participants aged >4 years, treated with miglustat and without double functional null mutations

Forty-one participants entered the OLE (arimoclomol, n=26; placebo, n=15); 33 completed M36. At M36, sustained benefit of arimoclomol was observed on the 5D-NPCCSS, with a mean (SD) change from baseline of 3.5 (5.6) for the arimoclomol-arimoclomol group (3 years' arimoclomol treatment; DB and OLE) and 0.9 (2.1) for the placebo-arimoclomol group (2 years' arimoclomol treatment; OLE only). In comparison, NPC progression with only standard of care was 5.2 (SE: 1.41) after 3 years and 3.5 (0.94) after 2 years, as estimated by extrapolation from NPC-001 observational and NPC-002 placebo data. A consistent benefit of sustained arimoclomol treatment was observed across subgroups at M36. Adverse events incidences at M12-24 (87.8%) and M24-36 (85.3%) were similar to the DB phase (88.2%).

Arimoclomol provided sustained treatment effect over 36 months; it was well tolerated, with a consistent safety profile.

### EQUIVALENTS

The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference.

The foregoing description has been presented only for the purposes of illustration and is not intended to limit the disclosure to the precise form disclosed, but by the claims appended hereto.

1.-13. (canceled)

14. A method of treating or preventing Niemann Pick disease, type C (NPC) in a subject in need thereof, the method comprising administering a therapeutically effective amount of arimoclomol to the subject, wherein the subject is identified as having at least one missense mutation in at least one of the two alleles in an NPC gene.

15. The method of claim 14, said method further comprising administering a therapeutically effective amount of miglustat to the subject.

16. A method of predicting the responsiveness of a subject with Niemann Pick disease, type C (NPC) to treatment with arimoclomol, the method comprising:
a) determining the presence or absence of at least one missense mutation in at least one allele of an NPC gene; and
b) predicting that the subject will respond to treatment with arimoclomol when the presence of at least one missense mutation in at least one allele of an NPC gene is determined.

17. A method of predicting the responsiveness of a subject with Niemann Pick disease, type C (NPC) to treatment with a combination of arimoclomol and miglustat, the method comprising:
a) determining the presence or absence of at least one missense mutation in at least one allele of an NPC gene; and;
b) predicting that the subject will respond to treatment with a combination of arimoclomol and miglustat when the presence of at least one missense mutation in at least one allele of an NPC gene is determined.

18.-19. (canceled)

20. The method of claim 14, the method comprising:
a) determining the presence or absence of at least one missense mutation in at least one allele of an NPC gene; and
b) administering a therapeutically effective amount of arimoclomol to the subject when the presence of at least one missense mutation in at least one allele of an NPC gene is determined.

21. The method of claim 20, the method comprising:
administering a therapeutically effective amount of miglustat to the subject.

22. The method of claim 14, wherein the subject is identified as having at least one missense mutation in each of the two alleles of the NPC gene.

23. The method of claim 14, wherein the subject is identified as having at least one missense mutation in one allele of the NPC gene and a functional null mutation in the other allele of the NPC gene.

24. (canceled)

25. The method of claim 14, wherein the subject is identified as being a compound heterozygote for the NPC gene.

26. The method of claim 14, wherein the NPC gene is NPC1.

27.-30. (canceled)

31. The method of claim 23, wherein the functional null mutation is a frameshift mutation, and wherein the frameshift mutation is selected from Q991fs, D508fs, H641fs, A1108fs, L1204fs, Q119fs, P733fs, L773fs, K822fs, I962fs, A1192fs, and L1248fs.

32. (canceled)

33. The method of claim 23, wherein the functional null mutation is an aberrant splicing mutation, and wherein the aberrant splicing mutation is selected from R518Q/sp, V20sp, G319sp, F1199sp1, and F1199sp2

34. The method of claim 23, wherein the functional null mutation is a premature stop codon mutation, wherein the premature stop codon mutation is selected from R116*, L860*, R934*, and E1188*.

35.-36. (canceled)

37. The method of claim 14, wherein the missense mutation is selected from I1061T, P1007A, S940L, R1186H, G886V, R978C, P1007L, L472P, R518W, G910S, A927V, S954L, G992W, S1004P, T1036M, N1156S, V1165M, N222S, G248V, S357L, R404W, E451K, Y677N, E718D, S734I, G765V, H897Q, A926V, N968S, P981L, M1001V, P1007R, H1016L, T1036A, A1054T, R1059Q, V1078I, F1079L, F1087L, V1141G, M1142T, A1151T, V1155G, and T1205K.

38.-41. (canceled)

42. The method of claim 23, wherein the functional null mutation is selected from D508fs, L773fs, K822fs, I962fs, F1199sp1, and F1199sp2.

43. (canceled)

44. The method of claim 26, wherein the subject has an NPC1 genotype selected from the group consisting of G886V/R978C, Y677N/R1059Q, E718D/P1007L, G765V/P1007A, A1054T/H897Q, P733fs/V1078I, T1036A/V1155G, D508fs/I1061T, G248V/L773fs, S734I/K822fs, I1061T/I962fs, V1165M/F1199sp1, and N1156S/F1199sp2.

45.-48. (canceled)

49. The method of claim 26, wherein the subject has an NPC1 genotype selected from the group consisting of I1061T/E1188*, I1061T/ A1151T, I1061T/Q119fs, I1061T/I962fs, T1036M /I1061T, I1061T/V1141G, N968S/ R1186H, N1156S /F1199sp2, Q991fs/I1061T, H1016L /I1061T, I1061T/A1192fs, I1061T/ N1156S, R1186H/R1186H, P1007A/R1186H, and I1061T/D508fs.

50.-60. (canceled)

61. The method of claim 14, wherein arimoclomol is the arimoclomol citrate salt administered at about 100 mg/day, about 125 mg/day, about 150 mg/day, about 175 mg/day, about 200 mg/day, about 225 mg/day, about 250 mg/day, about 275 mg/day, about 300 mg/day, about 325 mg/day, about 350 mg/day, about 375 mg/day, about 400 mg/day, about 425 mg/day, about 450 mg/day, about 475 mg/day, about 500 mg/day, about 525 mg/day, about 550 mg/day, about 575 mg/day, about 600 mg/day, about 625 mg/day, about 650 mg/day, about 675 mg/day, or about 700 mg/day.

62.-67. (canceled)

68. The method of claim 14, wherein arimoclomol is administered in a dosage adjusted by patient body weight.

69.-83. (canceled)

84. The method of claim 14, wherein arimoclomol is the citrate salt.
